(19) United States
(12) Patent Application Publication
(10) Pub. No.: US 2006/0019335 A1
(43) Pub. Date: Jan. 26, 2006

(54) Compositions and methods to promote c-IAP autodegradation

(76) Inventors: Chunying Du, Leawood, KS (US); Qi-Heng Yang, New York, NY (US)

Correspondence Address: POLSINELLI SHALTON WELTE SUELTHAUS P.C., 700 W. 47TH STREET, SUITE 1000, KANSAS CITY, MO, 64112-1802, US

(21) Appl. No.: 10/894,592

(22) Filed: Jul. 20, 2004

(51) Int. Cl. C12P 21/06

(52) U.S. Cl. 435/69.1

(57) ABSTRACT

The present invention generally relates to a method that may be employed as a means to regulate apoptosis. More particularly, the current invention relates to the use of Smac nucleic acid sequences and amino acid sequences expressed therefrom, which promote the ubiquitylation-mediated, auto-degradation of c-IAP.

### FIELD OF THE INVENTION

The present invention generally provides methods and compositions that may be employed as a means to regulate apoptosis. More particularly, the current invention relates to the use of Smac nucleotide sequences and amino acid sequences expressed therefrom, which promote the ubiquitylation-mediated, auto-degradation of c-IAP.

### BACKGROUND OF THE INVENTION

Apoptosis, known as programmed cell death, is an evolutionarily conserved and genetically regulated, biological process that is crucial for the normal development and homeostasis of multi-cellular organisms. In addition to genetically controlled programmed cell death, apoptosis can also be induced by cytotoxic lymphocytes, anti-cancer drugs, irradiation, by a group of cytokines known as death factors, and by deprivation of survival factors.

Aberrant regulation of apoptosis has been pathogenically linked to a plethora of human diseases. For example, cancer, autoimmune diseases, and neurodegenerative diseases are among the diseases linked to dysregulation of apoptosis. Due to the devastating consequences of dysregulation of apoptosis, methods and compositions that may be employed as a means to regulate apoptosis remain an unmet need.

Among the key regulators of apoptosis is a family of highly conserved, aspartate-specific, cysteine proteases known as caspases. Caspases are synthesized as inactive zymogens. But upon their activation, caspases are powerful facilitators of apoptosis. In fact, caspases cause the characteristic morphological changes associated with apoptotic cells. These morphological changes include chromatin condensation, DNA fragmentation into nucleosomal fragments, nuclear membrane breakdown, externalization of phosphotidylserine, and formation of apoptotic bodies that are readily phagocytosed.

Because of the pivotal role played by activated caspases in mediating apoptosis, a key means to regulate apoptosis is via the regulation of caspase activation. There are two known signal pathways leading to the activation of caspases, the death receptor pathway and the mitochondrial pathway. In the mitochondrial pathway, caspase activation is triggered by cytocrome c, a protein that normally functions in the electron transfer chain in mitochondria. In living cells, holocytochrome c is located exclusively in the intermembrane space of the mitochondria; and therefore, is sequestered away from its deadly cytosolic partner, Apaf-1. Upon receiving apoptotic stimuli, such as serum deprivation, activation of cell surface death receptors, or excessive damage of DNA, the outer membrane of mitochondria becomes permeable to cytochrome c. Once released to the cytosol, cytochrome c binds to Apaf-1 with 2:1 stoichiometry and forms an oligomeric Apaf-1/cytochrome c complex in the presence of dATP or ATP. In turn, this oligomerized Apaf-1/cytochrome c complex then recruits and activates the apical caspase of this pathway, procaspase-9. Caspase-9, in turn, activates downstream caspases, such as caspase-3, -6, and -7, which constitute the major caspase activity in an apoptotic cell.

A key regulator of caspases activation is the inhibitor of apoptosis proteins (IAPs). While first identified in baculvirises, IAPs have since been found in diverse species ranging from insects to humans. There are eight known mammalian IAPs that have been characterized including c-IAP1, cIAP2, XIAP, and livin. The apoptotic inhibitory activity of IAPs has been attributed to their ability to bind to and inhibit caspases, activated caspases, or procaspases. IAPs bind to caspases through an approximately 70 amino acid region on the N-terminal of the IAP molecule known as a BIR domain (Baculovirus IAP Repeat domain). Generally speaking, IAPs contain from one to three BIR domains, and each BIR domain has been shown to be functionally distinct. For example, it is known that BIR2 and BIR3 of XIAP are the domains that bind and inhibit activated caspases. The BIR2 domain of XIAP inhibits activated caspase 3, while the BIR3 domain of XIAP specifically inhibits activated caspases 9. The ability to modulate binding of the IAP to caspases through its BIR domain, accordingly, provides an attractive means to regulate apoptotic activity.

In addition to their BIR domains, a number of IAPs, including c-IAP1, c-IAP2 and XIAP, have another functionally active structural region that mediates their ability to regulate apoptosis. This region, a RING zinc-binding motif at the C-terminus, functions as an ubiquitin ligase toward target proteins. Ubiquitin-ligase enzymes, such as IAP, add ubitquitins to target proteins disposed with particular degradation signals, such as a free α-NH₂ having Arg, Lys, His, Phe, Tyr, Trp, Leu, Gln, Asp, or Glu at the N-terminus of the protein. After a certain number of additional ubiquitins are added, a poly-ubiquitin chain is formed on the target protein. This chain, in turn, is recognized by proteosomes, which then systematically degrade the target protein. While it remains to be fully elucidated how the auto-ubiquitination and degradation of IAPs are regulated, this facet of IAP function provides yet another means to regulate apoptotic activity.

One means to counteract the caspase-inhibiting activities of IAPs and promote apoptosis is via the application of an IAP antagonist protein. Examples of IAP antagonist proteins include Reaper, Hid, Grim, Sickle and Jafrac2 in the Drosophila, and Omi/HtrA2 and GSPT1/eRF in mammals. Despite the overall sequence differences, these IAP antagonists share a conserved N-terminal IAP-binding motif (IBM). The IBM functions to mediate the binding of the IAP antagonist protein to the IAP through its BIR domain. By binding to the BIR domain of the IAP, the IAP antagonist prevents the IAP from binding to caspases binding sites. Through this binding interaction, therefore, IAP antagonists abrogate the caspase inhibitory activity of IAPs and thereby potentiate apoptosis.

The second mitochondria derived activator of caspases, more commonly known as Smac, is a mammalian IAP antagonist that promotes cytochrome c/Apaf-1-dependent caspase activation. Like cytochrome c, Smac is normally located in the mitochondria and is released to the cytosol when cells undergo apoptosis. Similar to other IAP antagonists, Smac facilitates apoptosis by out competing IAP for caspases binding sites. Recently, however, another mechanism of action for a novel isoform of the Smac protein was elucidated. This isoform, named Smac3, which is generated by alternative splicing of exon 4 in Smac, was shown to accelerate XIAP auto-ubiquitination and destruction (Fu et al., (2003) J Biol Chem 278:52660-52672). Strikingly, Smac was not able to accelerate XIAP auto-ubiquitination and Smac3 was unable to cause this action in other mammalian IAPs.

Because different mammalian IAPs are differentially expressed in various cell types and in response to different disease states, such as cancer, a means to selectively target certain IAPs for auto-ubiquitination would provide a valuable tool to regulate apoptosis in a selected cell population. For example, if certain mammalian cancer cells over express XIAP relative to c-IAP, then a means to selectively facilitate XIAP for auto-ubiquitination (such as by administration of Smac3) would provide a mechanism to selectively target the cancer cells for apoptosis. In particular, this provides a template from which to design small molecules or drugs designed to promote apoptosis in a specific cell population. While a means to promote the auto-ubiquitination of XIAP has been partially determined, a means to promote the auto-ubiquitination of other mammalian IAP has yet to be elucidated. In particular, a means to selectively promote c-IAP autodegradation remains an unmet need.

### SUMMARY OF THE INVENTION

Among the several aspects of the invention, accordingly, is provided a method and a composition for promoting the autodegradation of c-IAP, specifically c-IAP1 and c-IAP2. Advantageously, the method may be employed to selectively promote the autodegradation of c-IAP in a medium containing a plurality of different IAPs. The method may also be utilized as a means to facilitate caspase 8 activation and ultimately to regulate apoptosis. Smac having SEQ ID NO: 1 is a polypeptide or protein that promotes autodegradation of c-IAP in mammalian cells.

Briefly, therefore, one aspect of the present invention encompasses a method for promoting the auto-degradation of an isolated c-IAP. The method comprises contacting in vitro the c-IAP with an effective amount of an isolated Smac polypeptide. Upon contact, the Smac polypeptide binds to the c-IAP and enhances the auto-ubiquitylation activity of the c-IAP. Due to this increased auto-ubiquitylation activity, c-IAP auto-degradation is promoted.

The present invention is further directed to such a method in which the c-IAP is selectively auto-degraded. In this embodiment, the method comprises contacting in vitro a medium having a plurality of IAP molecules and an effective amount of Smac polypeptide. Upon contact, the Smac polypeptide binds to the c-IAP present in the medium and enhances the auto-ubiquitylation activity of the c-IAP. Due to this increased auto-ubiquitylation activity, c-IAP auto-degradation is selectively promoted relative to other IAPs present in the medium.

A further aspect of the invention provides a method to promote the auto-degradation of a c-IAP in a cell. The method comprises introducing into the cell an effective amount of a Smac polypeptide. Depending upon the embodiment, the Smac polypeptide may be derived from a variety of sources, including nucleotide sequences encoding a Smac polypeptide; a vector comprising a nucleotide sequence encoding a Smac polypeptide; and, an isolated Smac polypeptide. Upon introduction into the cell, the Smac polypeptide is contacted with and binds to the c-IAP present in the cell, thereby enhancing the auto-ubiquitylation activity of the c-IAP. Due to this increased auto-ubiquitylation activity, c-IAP auto-degradation is promoted.

The present invention is further directed to such a method in which the c-IAP is selectively auto-degraded in the cell. In this embodiment, the method comprises introducing a Smac polypeptide (derived from any of the sources delineated above) into a cell having a plurality of IAP molecules. Upon introduction into the cell, the Smac polypeptide is contacted with and binds to the c-IAP present in the cell and enhances the auto-ubiquitylation activity of the c-IAP. Due to this increased auto-ubiquitylation activity, c-IAP auto-degradation is selectively promoted relative to other IAP molecules present in the cell.

In still another aspect of the invention is provided a method to promote the E₃ activity of a c-IAP molecule. The method comprises contacting in vitro the c-IAP molecule with an isolated Smac polypeptide. Upon contact, the Smac polypeptide binds to, and functions to stimulate the E₃ activity of c-IAP

Another aspect of the invention provides an in vitro composition. The composition comprises an isolated Smac polypeptide, c-IAP degradation products and E₃. The composition is typically suspended in a carrier.

Yet another aspect of the invention is an isolated ubiquilated molecule. The molecule comprises a Smac polypeptide, c-IAP, ubiquitin, and a proteosome.

Other aspects and embodiments of the invention are further described herein.

### BRIEF DESCRIPTION OF THE DRAWINGS

FIG. 1 shows the ability of the Smac polypeptide to reduce the protein level of c-IAP1 and c-IAP2 in HeLa cells;

FIG. 2A shows the ability of Smac polypeptide to promote the rapid degradation of c-IAP1 by enhancing its auto-ubiquitylation;

FIG. 2B shows the ability of Smac polypeptide to promote the rapid degradation of c-IAP2 by enhancing its auto-ubiquitylation;

FIG. 3A shows the ability of Smac polypeptide to promote auto-ubiquitylation but not degradation of XIAP;

FIG. 3B shows the ability of Smac polypeptide to promote auto-ubiquitylation but not degradation of Livin;

FIG. 4 shows that IAP proteins bind the Smac WT polypeptide, but not Smac ΔA mutant polypeptide;

FIGS. 5A and 5B show that different IAP proteins require the same ubiquitin-conjugating enzymes for their E3 activity in vitro;

FIG. 6 shows that Smac polypeptide does not promote the auto-ubiquitylation of XIAP in vitro; and,

FIGS. 7A and 7B show that Smac polypeptide promotes the auto-ubiquitylation of c-IAP1 in vitro.

### DETAILED DESCRIPTION OF THE INVENTION

The present invention generally provides methods and compositions that may be employed as a means to regulate apoptosis. More particularly, the current invention relates to the use of isolated Smac polypeptide to promote the ubiquitylation-mediated, auto-degradation of c-IAP. The degradation of c-IAP, in turn, facilitates caspase activation, which in turn, will lead to apoptosis in a cell. Generally speaking, the method of the invention comprises contacting, either in vivo or in vitro, c-IAP with an effective amount of an isolated Smac polypeptide. Upon contact, the Smac polypeptide binds to the c-IAP and enhances the auto-ubiquitylation activity of the c-IAP. Due to this increased auto-ubiquitylation activity, c-IAP auto-degradation is promoted.

Smac Polypeptides

A Smac polypeptide suitable for use in the present invention is one that functionally will bind to c-IAP and as a result, will enhance the auto-ubiquitylation activity of the c-IAP; specifically, it will bind to c-IAP1 or c-IAP2. In a typical embodiment, the Smac polypeptide may functionally bind to one or more species of IAP proteins, including c-IAP, but will selectively enhance the auto-ubiquitylation activity of the c-IAP compared to other IAP proteins to which the Smac polypeptide will also bind. This ability of a Smac polypeptide to selectively cause c-IAP auto-ubiquitylation relative to either XIAP or Livin auto-ubiquitylation is illustrated in the Examples herein. Methods to determine whether a particular Smac polypeptide causes selective auto-ubiquitylation of c-IAP is also described in the Examples.

In one aspect of the invention, the isolated Smac polypeptide is a polypeptide having SEQ ID NO 1. The Smac polypeptide corresponding to SEQ ID NO 1 is the wild type (WT) Smac polypeptide isolated from Homo sapiens (humans). Similar polypeptides are, for example, Reaper, Hid, Grim, Sickle and Jafrac2 in the Drosophila, and Omi/HtrA2 and GSPT1/eRF in mammals. The Smac polypeptide having SEQ ID NO 1 is a polypeptide that is 239 amino acids in length and has an N-terminal hexapeptide corresponding to the amino acids AVPIAQ (SEQ ID NO 2). This hexapeptide, referred to herein as Smac-6, functions to stimulate the E₃ activity of c-IAP. In fact, as illustrated in the Examples, Smac-6 stimulates the E₃ activity of c-IAP as effectively as the full length Smac polypeptide having SEQ ID NO 1. The polypeptide of SEQ ID NO 1 also includes an N-terminal IAP-binding motif that is conserved across a number of IAP antagonists, including the Drosophila polypeptides Grim, Hid, and Reaper.

In certain aspects, polypeptides that are homologs or degenerative variants of the polypeptide having SEQ ID NO 1 are also suitable for use in the present invention. Typically, the subject polypeptides include fragments that share substantial sequence similarity, binding specificity and function with the polypeptide having SEQ ID NO 1. In particular, the polypeptide will have a substantially similar biological function as the polypeptide having SEQ ID NO 1. This biological activity includes stimulating the E₃ activity of c-IAP, whereby the enhanced E₃ activity results in the auto-ubiquitylation mediated auto-degradation of the c-IAP. Moreover, the homolog or degenerative variant will also preferably selectively enhance the auto-ubiquitylation activity of the c-IAP compared to other IAP proteins to which the Smac polypeptide will also bind.

A number of methods may be employed to determine whether a particular homolog or degenerative variant possesses substantially similar biological activity relative to the Smac polypeptide having SEQ ID NO 1. Specific activity or function may be determined by convenient in vitro, cell-based, or in vivo assays, such as, in vitro binding assays. Binding assays encompass any assay where the molecular interaction of a subject polypeptide with a binding target is evaluated. The binding target may be a natural binding target such as a regulating protein or a non-natural binding target such as a specific immune protein such as an antibody, or a specific agent such as those identified in screening assays. Binding specificity may be assayed by binding equilibrium constants (usually at least about 10⁷ M⁻¹, preferably at least about 10⁸ M⁻¹, more preferably, at least about 10⁹ M⁻¹), by caspase activation or apoptosis assays, or by the ability of the subject polypeptide to function as negative mutants in expressing cells. In order to determine whether a particular polypeptide selectively promotes the autodegradation of c-IAP, the procedures detailed in the examples may be followed.

In addition to having a substantially similar biological function, a homolog or degenerative variant suitable for use in the invention will also typically share substantial sequence similarity to the Smac polypeptide and SEQ ID NO 1. Generally speaking, the subject polypeptide will have an N-terminal hexapeptide comprising SEQ ID NO 2 and will also have an N-terminal IAP-binding motif. In addition, suitable homologs or degenerative variants preferably share at least 50% sequence homology with SEQ ID NO 1, more preferably, 75%, and even more preferably, are greater than about 90% homologous in sequence to SEQ ID NO 1. Typically, sequence differences between a selected homolog or variant and SEQ ID NO 1 will include a number of conservative amino acid substitutions. A “conservative substitution” is a substitution that does not abolish the ability of the Smac polypeptide to promote the selective autodegradation of c-IAP, as described herein.

In determining whether a polypeptide is substantially homologous to Smac polypetide, sequence similarity may be determined by conventional algorithms, which typically allow introduction of a small number of gaps in order to achieve the best fit. In particular, “percent homology” of two polypeptides or two nucleic acid sequences is determined using the algorithm of Karlin and Altschul (Proc. Natl. Acad. Sci. USA 87:2264-2268, 1993). Such an algorithm is incorporated into the NBLAST and XBLAST programs of Altschul et al. (J. Mol. Biol. 215:403-410, 1990). BLAST nucleotide searches may be performed with the NBLAST program to obtain nucleotide sequences homologous to a nucleic acid molecule of the invention. Equally, BLAST protein searches may be performed with the XBLAST program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., XBLAST and NBLAST) are employed. See http://www.ncbi.nlm.nih.gov for more details.

Smac polypeptides suitable for use in the invention are isolated or pure. An “isolated” polypeptide is unaccompanied by at least some of the material with which it is associated in its natural state, preferably constituting at least about 0.5%, and more preferably, at least about 5% by weight of the total polypeptide in a given sample. A pure polypeptide constitutes at least about 90%, preferably, 95% and even more preferably, at least about 99% by weight of the total polypeptide in a given sample.

The Smac polypeptide may be synthesized, produced by recombinant technology, or purified from cells. In one embodiment, the Smac polypeptide of the present invention may be obtained by direct synthesis. In addition to direct synthesis, the subject polypeptides can also be expressed in cell and cell-free systems (e.g. Jermutus L, et al., Curr Opin Biotechnol. October 1998; 9(5):534-48) from encoding polynucleotides, such as from SEQ ID NO 3 (as described below) or naturally-encoding polynucleotides isolated with degenerate oligonucleotide primers and probes generated from the subject polypeptide sequences (“GCG” software, Genetics Computer Group, Inc, Madison Wis.) or polynucleotides optimized for selected expression systems made by back-translating the subject polypeptides according to computer algorithms (e.g. Holler et al. (1993) Gene 136, 323-328; Martin et al. (1995) Gene 154, 150-166). In other embodiments, any of the molecular and biochemical methods known in the art are available for biochemical synthesis, molecular expression and purification of the Smac polypeptides, see e.g. Molecular Cloning, A Laboratory Manual (Sambrook, et al. Cold Spring Harbor Laboratory), Current Protocols in Molecular Biology (Eds. Ausubel, et al., Greene Publ. Assoc., Wiley-Interscience, New York).

Smac Nucleotide Sequences

The present invention also encompasses the use of an isolated Smac nucleotide sequences. In particular, the subject nucleotide sequences may be utilized as a means to produce a Smac polypeptide having the structure and biological activity as detailed above.

In one aspect, the Smac WT gene is employed. The Smac WT gene corresponds to SEQ ID NO 3. The gene is isolated from Homo sapiens and is comprised of 1358 nucleic acids. This gene encodes the Smac polypeptide having SEQ ID NO 1.

The invention also encompasses the use of nucleotide sequences other than SEQ ID NO 3 that encode Smac polypeptides having the structure and function described above. Typically, these nucleotide sequences will hybridize under stringent hybridization conditions (as defined herein) to all or a portion of the nucleotide sequence represented by SEQ ID NO 3 or its complement. The hybridizing portion of the hybridizing nucleic acids is usually at least 15 (e.g., 20, 25, 30, or 50) nucleotides in length. The hybridizing portion of the hybridizing nucleic acid is at least 80%, preferably, at least 90%, and is more preferably, at least 95% identical to the sequence of a portion or all of a nucleic acid sequence encoding a Smac polypeptide suitable for use in the present invention, or its complement.

Hybridization of the oligionucleotide probe to a nucleic acid sample, such as SEQ ID NO 3, is typically performed under stringent conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Then, assuming at 1% mismatching results in a 1° C. decrease in the Tm, the temperature of the final wash in the hybridization reaction is reduced accordingly. For example, if sequences have greater than 95% identity with the probe is sought, the final temperature is approximately decreased by 5° C. In practice, the change in Tm can be between 0.5 and 1.5° C. per 1% mismatch. Stringent conditions involve hybridizing at 68° C. in 5×SSC/5× Denhardt's solution/1.0% SDS, and washing in 0.2×SSC/0.1% SDS at room temperature. Moderately stringent conditions include washing in 3×SSC at 42° C. The parameters of salt concentration and temperature can be varied to achieve the optimal level of identity between the probe and SEQ ID NO 3. Additional guidance regarding such conditions is readily available in the art, for example, by Sambrook et al., 1989, Molecular Cloning, A Laboratory Manual, Cold Spring Harbor Press, N.Y.; and Ausubel et al., (eds.), 1995, Current Protocols in Molecular Biology, (John Wiley & Sons, N.Y.) at Unit 2.10.

The various nucleic acid sequences mentioned above can be obtained using a variety of different techniques known in the art. The Smac WT nucleic acid sequence (i.e. SEQ ID NO 3), as well as homologous sequence encoding a suitable Smac polypeptide, can be isolated using standard techniques, or can be purchased or obtained from a depository. Once the Smac WT nucleotide sequence is obtained, it can be amplified for use in a variety of applications, as further described below.

Construction of Vectors Encoding Smac Nucleotide Sequences

Once selected in accordance with the above criteria, the isolated Smac nucleic acid sequences can be placed into various vectors, such as expression vectors, fusion vectors, gene therapy vectors, two-hybrid vectors, reverse two-hybrid vectors, sequencing vectors, and cloning vectors. The vectors can include activator or promoter sequences, as well as markers. An inducible promoter may also be included in the vector. The resultant vector will include a suitable Smac nucleic acid sequence and, optionally, a marker or activator. It is preferred to include a promoter as well. Suitable vectors will include those vectors that Smac nucleic acid sequences can be inserted into and resultantly, can be employed to introduce Smac polypeptide into a target cell. A number of examples of suitable vectors are described below.

Selectable marker genes may be introduced into vectors by a number of methods commonly known in the art. Typically, a selectable marker will be used to ensure that a targeted nucleic acid sequence has been incorporated into the vector. There are three general categories of selectable marker genes available, including antibiotic resistant marker genes, metabolic/auxotrophic marker genes, and screenable marker genes.

In one embodiment, the selectable maker is an antibiotic resistant marker. Antibiotic resistant marker genes confer the phenotypic trait of resistance to a specific antibiotic. For example, the neomycin phosphotransferase II (NPT II) gene is a selectable marker for resistance to the antibiotics neomycin and kanamycin.

In an alternative embodiment, the selectable marker is a metabolic maker. Metabolic or auxotrophic marker genes enable transformed cells to synthesize an essential component, usually an amino acid, which the cells cannot otherwise produce. The cell culture medium is made to intentionally lack the essential component, which cells require for growth. Cells that have successfully incorporated the selectable marker can readily be distinguished from ones that haven't incorporated the marker because they are able to survive and grow in the component-deficient medium. These cells can be selected and regenerated into whole mutant organisms.

In yet another alternative embodiment, the selectable marker is a screenable marker. Sreenable markers, also known as assayable markers, are genes that encode a protein that can then be readily identified through other laboratory methods. The presence of the protein confirms that transformation has taken place. Examples of suitable screenable markers, which are epitope tags, include HIS, MYC, HA, HSV, V5, and FLAG. These sequences encode short peptides that create an antigenic determinant (epitope) that can be recognized by antibodies. Thus, when the DNA sequence of interest is linked with the DNA sequence of the short peptide, the resulting exported protein is now a “tagged” protein. Since antibodies to the peptide tag are readily available commercially, immunoprecipitation or immunopurification of the tagged fusion proteins can be readily accomplished.

Introduction of Smac Nucleotide Sequences and Polypeptides into Cells

One aspect of the current invention encompasses introduction of isolated Smac nucleotide sequences and polypeptide sequences into a target cell for the purpose of protein production. A number of methods are suitable for such introduction and will vary depending upon the particular sequence and target cell. Generally speaking, the cell may be an in vivo or in vitro cell. For example, the Smac nucleotide sequences can be expressed by a recombinant cell, such as a bacterial cell, a cultured eukaryotic cell, or a cell disposed in a living organism, including a non-human transgenic organism, such as a transgenic animal. By way of non-limiting example, cultured cells available for use include Hela cells, HEK 293 cells and U937 cells, as well as other cells used to express proteins. A preferred cell is a mammalian cell, and more preferably, a human tumor cell. Exemplary cells include, for example, tumor cells, such as leukemic or carcinoma cells, or heart cells.

In one embodiment of the invention, a vector, such as a vector detailed above, can be employed to introduce a suitable Smac polynucleotide into a host cell. Typically, in this aspect of the invention, the Smac polynucleotide is incorporated into an expression vector, which subsequently is utilized to transfect a target cell. Depending upon the embodiment, the cell may be a cultured cell or a cell disposed within a living organism. Irrespective of the embodiment, the vector binds to the target cell membrane, and the Smac nucleotide sequence is internalized into the cell. The vector comprising the Smac nucleotide sequence may be either integrated into the target cell's nucleic acid sequence or may be a plasmid. Irrespective of its form, the vector employed results in Smac polypeptide expression. As such, a suitable vector for the present invention is one that can transfect a desired cell, and effectively deliver a Smac nucleotide sequence that results in expression of a functional Smac polypeptide having the properties detailed above. There are a number of suitable expression transfer vectors that may be utilized in the practice of the invention. By way of non-limiting example, these include eukaryotic gene transfer expression vectors including retroviruses, adenoviruses, adeno-associated viruses, and herpes viruses.

In one embodiment, the transfer vector is a retrovirus. Retroviruses can package up to 5 Kb of exogenous nucleic acid material, and can efficiently infect dividing cells via a specific receptor, wherein the exogenous genetic information is integrated into the target cell genome. In the host cell cytoplasm, the reverse transcriptase enzyme carried by the vector converts the RNA into proviral DNA, which is then integrated into the target cell genome, thereby expressing the transgene product (i.e., the Smac polypeptide).

In another alternative embodiment, the transfer vector is an adenovirus. In general, adenoviruses are large, double-stranded DNA viruses which contain a 36 Kb genome that consists of genes encoding early regulatory proteins and a late structural protein gene. Adenoviruses, advantageously, can be grown in high titers of purified recombinant virus (up to 10¹² infectious particles/ml), incorporate large amounts of exogenous genetic information, and can broadly infect a wide range of differentiated non-dividing cells in vivo.

In yet another alternative embodiment, the transfer vector is an adeno-associated virus (AAV). AAV is a human parvovirus that is a small, single-stranded DNA virus that can infect both dividing and non-dividing cells. AAV is relatively non-toxic and non-immunogenic and results in long-lasting expression. The packaging capacity of recombinant AAV is 4.9 kb. Successful AAV-mediated gene transfer into brain, muscle, heart, liver, and lung tissue has been reported.

Exemplary transfer vectors for transfer into eukaryotic cells include MSCV, Harvey murine sarcoma virus, pFastBac, pFastBac HT, pFastBac DUAL, pSFV, pTet-Splice, pEUK-Cl, pPUR, pMAM, pMAMneo, pBI101, pBI121, pDR2, pCMVEBNA, YACneo, pSVK3, pSVL, pMSG, pCH110, pKK232-8, p3'SS, pBlueBacIII, pCDM8, pcDNA1, pZeoSV, pcDNA3, pREP4, pET21b, pCEP4, and pEBVHis vectors. Most preferably, the MSCV virus can be used.

The transfected cells include isolated in vitro population of cells. In vivo, the vector can be delivered to selected cells, whereby the carrier for the vector is attracted to the selected cell population.

Activation of the gene in a transfected cell can be caused by an external stress factor. For example, the transfected cells can be contacted with an etoposide or a proteosome inhibitor. In the alternative, an activator can be included in the vector in accordance with the methods detailed above.

In another alternative embodiment, the Smac nucleotide sequence can be introduced into a target cell by mechanical, electrical or chemical procedures. Mechanical methods include microinjection, pressure, and particle bombardment. Electrical methods include electroporation. Chemical methods include liposomes, DEAE-dextran, calcium phosphate, artificial lipids, proteins, dendrimers, or other polymers, including controlled-release polymers.

In one aspect of this embodiment, accordingly, a mechanical method is employed to introduce Smac nucleotide sequences into the target cell. One such method is hydrodynamic force and other external pressure-mediated DNA transfection methods. Alternatively, ultrasonic nebulization can be utilized for DNA-lipid complex delivery. In other suitable embodiments, particle bombardment, also known as biolistical particle delivery, can be utilized to introduce DNA into several cells simultaneously. In still another alternative mechanical method, DNA-coated microparticles (e.g., gold, tungsten) are accelerated to high velocity to penetrate cell membranes or cell walls. This procedure is used predominantly in vitro for adherent cell culture transfection.

In a further aspect of this embodiment, an electrical method is employed to introduce Smac nucleotide sequences into the target cell. In one alternative of this embodiment, electroporation is employed. Electroporation uses high-voltage electrical impulses to transiently permeabilize cell membranes, and thereby, permits cellular uptake of macromolecules, such as nucleic acid and polypeptide sequences.

In an additional aspect of this embodiment, a chemical method is employed to introduce Smac nucleotide sequences into the target cell. Chemical methods, using uptake-enhancing chemicals, are highly effective for delivering nucleic acids across cell membranes. For example, nucleotide sequences are typically negatively charged molecules. DEAE-dextran and calcium phosphate, which are positively charged molecules, interact with nucleotide sequences to form DEAE-dextran-DNA and calcium phosphate-DNA complexes, respectively. These complexes are subsequently internalized into the target cell by endocytosis.

In another alternative embodiment, the chemical enhancer is lipofectin-DNA. This complex comprises an artificial lipid-based DNA delivery system. In this embodiment, liposomes (either cationic, anionic, or neutral) are complexed with DNA. The liposomes can be used to enclose a Smac nucleic acid for delivery to target cells, in part, because of increased transfection efficiency.

In yet another alternative chemical embodiment, protein-based methods for DNA introduction may also utilized. The cationic peptide poly-L-lysine (PLL) can condense DNA for more efficient uptake by cells. Protamine sulfate, polyamidoamine dendrimers, synthetic polympers, and pyridinium surfactants may also be utilized.

In still a further chemical embodiment for Smac nucleotide introduction, biocompatible controlled-release polymers may be employed. Biodegradable poly (D,L-lactide-co-glycolide) microparticles and PLGA microspheres have been used for long-term controlled release of DNA molecules to cells. In a further embodiment, Smac nucleotide sequences may also be encapsulated into poly(ethylene-co-vinyl acetate) matrices, resulting in long term controlled, predictable release for several months.

Similarly, as for the introduction of Smac nucleotide sequences, the Smac polypeptide can also be introduced into target cells by any of the mechanical, electrical or chemical means detailed above. Mechanical methods include microinjection, pressure, and particle bombardment. Direct microinjection of Smac polypeptide into cells in vitro occurs directly and efficiently. As with DNA-injected cells, once cells are modified in vitro, they can be transferred to the in vivo host environment. In particle bombardment, Smac polypeptide-coated microparticles are physically hurled with force against cell membranes or cell walls to penetrate cells in vitro. Electroporation, particularly at low voltage, and high frequency electrical impulses, is suitable for introduction of Smac polypeptides with in vitro or in vivo. Moreover, any of the chemical means detailed above may also be employed.

Irrespective of whether the sequence being introduced into the cell is a Smac nucleic acid or polypeptide, a suitable means for cellular introduction will possess the following properties: ease of packaging assembly of Smac sequence; delivery to target cells leading to high transfection efficiencies; stabilization of DNA molecules, bypassing or escaping from cellular endocytotic degradative pathways; efficient decomplexation or unpackaging of DNA upon intracellular release; efficient nuclear targeting of Smac DNA; and high, persistent, and controllable expression of therapeutic levels of Smac polypeptides.

Ubiguitylation of IAP Molecules

In several embodiments of the present invention, IAP molecules are subjected to ubiquitylation both in vitro and in vivo. “Ubiquitin” is a highly conserved 76 amino acid protein expressed in all eukaryotic cells. Generally speaking, as employed in the practice of the invention, ubiquitylation refers to a process that involves the covalent ligation of ubiquitin to a target protein, such as IAP, resulting in a poly-ubiquitinated target protein that is rapidly detected and degraded by the 26S proteasome. A cascade of enzymatic activity mediates the ubiquitination of a target protein. Ubiquitin is first activated in an ATP-dependent manner by a ubiquitin activating enzyme (E1). The C-terminus of a ubiquitin forms a high-energy thiolester bond with E1. The ubiquitin is then passed to a ubiquitin-conjugating enzyme (E2; also called ubiquitin carrier protein), also linked to this second enzyme via a thiolester bond. The ubiquitin is finally linked to its target protein to form a terminal isopeptide bond under the guidance of a ubiquitin ligase (E3). In this process, chains of ubiquitin are formed on the target protein, each covalently ligated to the next through the activity of E3.

Typically, a composition subjected to ubiquitylation will include ubiquitin, E1, E2, and E3. In a preferred embodiment, the ubiquitin is mammalian. In a more preferred embodiment, the ubiquitin is human. Other suitable ubiquitin proteins include, for example, those that may be made through the expression of the nucleic acid of ATCC accession number M26880 or U49869, each of which is incorporated herein by reference.

E1 proteins useful in the invention include those having the amino acid sequence of the polypeptide having ATCC accession numbers A38564, S23770, AAA61246, P22314, CAA40296 and BAA33144, all of which are incorporated herein by reference. In an alternative embodiment, nucleic acids may be used for producing E1 proteins for the invention include, but are not limited to, those disclosed by ATCC accession numbers M58028, X56976 and AB012190, incorporated herein by reference. E1 is also commercially available from Affiniti Research Products (Exeter, U.K.).

Compositions of the invention also generally comprise E2. By “E2” is meant a ubiquitin carrier enzyme (also known as a ubiquitin-conjugating enzyme). In one embodiment, ubiquitin is transferred from E1 to E2. In general, the transfer results in a thiolester bond formed between E2 and ubiquitin. Suitable E2 proteins that may be used in the present invention include, but are not limited to, those having the amino acid sequences disclosed in ATCC accession numbers AAC37534, P49427, CAA82525, AAA58466, AAC41750, P51669, AAA91460, AAA91461, CAA63538, AAC50633, P27924, AAB36017, Q16763, AAB86433, AAC26141, CAA04156, BAA11675, Q16781 and CAB45853, each of which is incorporated herein by reference. In a preferred embodiment, E2 is Ubc5c, UbCH5a, or UbCH6. In an alternative embodiment, nucleic acids may be used to make E2 and include, but are not limited to, those nucleic acids having sequences disclosed in ATCC accession numbers L2205, Z29328, M92670, L40146, U39317, U39318, X92962, U58522, S81003, AF031141, AF075599, AJ000519, and D83004, each of which is incorporated herein by reference.

The present invention also provides methods and compositions comprising E3. By “E3” is meant a ubiquitin ligase, as defined herein, comprising one or more components associated with ligation of ubiquitin to a ubiquitination substrate protein, such as IAP, for ubiquitin-dependent proteolysis. In a preferred embodiment, the E3 employed is as detailed in the examples.

In certain aspects, polypeptides that are homologues or degenerative variants of ubiquitin, E1, E2 and/or E3 may also be suitable for use in the present invention. Typically, the subject polypeptides include fragments of the recited sequence that have ubiquitin, E1, E2 and/or E3-specific amino acid sequence, binding specificity and function.

In a preferred embodiment, ubiquitin, E1, E2, and E3 may have a tag. Preferred tags include, but are not limited to, labels, partners of binding pairs and substrate binding elements. In a most preferred embodiment, the tag is a His-tag or GST-tag.

Ubiquitin, E1, E2, and E3 may be added to compositions of the invention in varying amounts, as can be readily determined by a skilled artisan and as illustrated in the examples herein. Generally speaking, the ubiquitylation components are combined under reaction conditions that favor ubiquitin ligase activity. Typically, therefore, this will be physiological conditions. But depending upon the embodiment, incubations may be performed at any temperature that facilitates optimal activity, typically between 4 and 40° C. Incubation periods are selected for optimum activity, but may also be optimized to facilitate rapid high through put screening. Typically between 0.5 and 1.5 hours will be sufficient.

A variety of other reagents may be included with the ubiquitylation components. These include reagents like salts, solvents, buffers, neutral proteins, e.g. albumin, detergents, etc. which may be used to facilitate optimal ubiquitination enzyme activity and/or reduce non-specific or background interactions. Also reagents that otherwise improve the efficiency of the assay, such as protease inhibitors, nuclease inhibitors, anti-microbial agents, etc., may be used. The compositions will also preferably include adenosine tri-phosphate (ATP).

The mixture of components may be added in any order that promotes ubiquitin ligase activity. In a preferred embodiment, ubiquitin is provided in a reaction buffer solution, followed by addition of the ubiquitination enzymes. In a particularly preferred embodiment, the ubiquitylation reaction is preformed as described in the examples herein.

Uses of the Smac Polypeptides and Nucleic Acid Sequences

As detailed herein, IAP proteins bind to and inhibit caspases activation via their IAP repeat (BIR) domains. Inhibition of caspases activation, concomitantly, results in an inhibition of apoptosis. It has been discovered that Smac polypeptide selectively causes the rapid auto-degradation of c-IAP, but not other IAP molecules, even though it binds to and promotes the auto-ubiquitylation of all of them. It has also been discovered that this selective degradation results in part from the Smac polypeptide's ability to enhance the E₃ activity of the c-IAP molecule. Taken together, the Smac polypeptide may be employed to reduce the protein level of c-IAP through the ubiquitin/proteosomal pathway.

In view of the above, one aspect of the invention provides a method for promoting the auto-degradation of an isolated c-IAP. The method comprises contacting in vitro the c-IAP with an effective amount of an isolated Smac polypeptide. Upon contact, the Smac polypeptide binds to the c-IAP and enhances the auto-ubiquitylation activity of the c-IAP. Due to this increased auto-ubiquitylation activity, c-IAP auto-degradation is promoted.

Yet another aspect of the invention is directed to such a method as described above in which the c-IAP is selectively auto-degraded. In this embodiment, the method comprises contacting in vitro a medium having a plurality of IAP molecules and an effective amount of Smac polypeptide. Upon contact, the Smac polypeptide binds to the c-IAP present in the medium and enhances the auto-ubiquitylation activity of the c-IAP. Due to this increased auto-ubiquitylation activity, c-IAP auto-degradation is selectively promoted relative to other IAPs present in the medium.

In each of the in vitro methods detailed above, the Smac polypeptide to c-IAP molar ratio is between about 1:5 to about 1:30. More typically, the molar ratio in vitro of Smac polypeptide to c-IAP is about 1:1. Depending upon the embodiment, the in vitro reaction parameters may vary considerably. Generally speaking, the in vitro conditions will include an incubation time of about 1 to about 3 hours at about 37° C. in solution. Moreover, the in vitro solution, depending on the embodiment, may include a plurality of different IAP molecules. The IAP molecule that is present is typically derived from cells selected from the group consisting of insects, mammalian, reptile, aves, and amphibian cells. In a preferred embodiment, the IAP molecule is from a mammal such as a human. By way of example, the IAP molecule may include c-IAP1, cIAP2, XIAP, Livin α, Livin β, and DIAP1.

A further aspect of the invention provides a method to promote the auto-degradation or the selective auto-degradation of a c-IAP in vivo, such as in a cell. The method comprises introducing into the cell an effective amount of a Smac polypeptide. Depending upon the embodiment, the Smac polypeptide may be derived from a source selected from the group consisting of: a nucleic acid sequence encoding a Smac polypeptide; a vector comprising a nucleic acid sequence encoding a Smac polypeptide; an isolated Smac polypeptide, or any other suitable means of cellular introduction described herein. Upon introduction into the cell, the Smac polypeptide is contacted with and binds to the c-IAP present in the cell, thereby enhancing the auto-ubiquitylation activity of the c-IAP. Due to this increased auto-ubiquitylation activity, c-IAP auto-degradation or selective auto-degradation is promoted.

By way of non-limiting example, in vivo promotion of c-IAP auto-degradation can be accomplished by transfecting a mammalian cell with a vector comprising a suitable Smac nucleic acid sequence and an appropriate activator. Once a population of cells has been transfected with a population of such vectors, Smac polypeptide expression can be stimulated by treating the cells with an etoposide, or similar composition, which causes the appropriate stress response to induce the activator (and results in stimulation of the activator).

In an alternative non-limiting example, the Smac polypeptide may be introduced in vivo via the use a carrier, such as a liposome, with a Smac polypeptide. The carrier, or liposome, will transport the Smac polypeptide across the cell membrane and place Smac polypeptide in contact with c-IAP. Again, upon contact, c-IAP auto-degradation will be promoted.

In still another aspect of the invention is provided a method to promote the E₃ activity of a c-IAP molecule. The method comprises contacting, either in vitro or in vivo, the c-IAP molecule with an isolated Smac polypeptide. If the method is in vivo, the Smac polypeptide may be introduced into the cell via any of the methods described herein.

The Smac polypeptide can be used as part of a method for reducing the level of c-IAP in a cell. This will, in turn, result in caspase activation. In a particularly preferred embodiment, caspase-8 activation may be facilitated. Thus, a method for promoting apoptosis and causing caspase activation, and in particular, caspase-8 activation, can be practiced via the methods of the invention.

In a particularly preferred embodiment, the methods may be employed to regulate apoptotic signaling mediated by the TNFR family. Generally speaking, while not being bound by any particular theory or mechanism, the Smac polypeptide, because of its ability to promote E₃ activity of c-IAP, may accelerate TNFR-mediated caspase activation in vivo by promoting such E3 activity and proteasomal degradation of c-IAP-1 and c-IAP-2. This theory is particularly practical in view of the fact that both c-IAP-1 and c-IAP-2 are known to be involved in the apoptotic signaling mediated by the TNRF family (33, 42-47). Moreover, Smac polypeptide is known to coexist with c-IAP1, TRAF2 and TRAF3 in an endogenous ligand-receptor complex (48). Further, it is known that Smac polypeptide makes c-IAP1 dissociate from TRAF2, which in turn releases the inhibition of caspases-8 activation by TRAF2-c-IAP1 complex (33). Taken collectively, Smac polypeptide may be utilized as a means to regulate TNFR-mediated caspase activation.

An in vitro composition will include an isolated Smac polypeptide, c-IAP degradation products and E₃. Alternatively, the composition may comprise an isolated Smac-6 polypeptide, c-IAP degradation products and E₃ The Smac polypeptide, c-IAP degradation products and E₃ include any of the species for each respective molecule described herein. Similarly, the Smac-6 polypeptide, c-IAP degradation products and E₃ include any of the species for each respective molecule described herein.

Yet another aspect of the invention is an isolated ubiquilated molecule. The molecule comprises a Smac polypeptide, c-IAP, ubiquitin, and a proteosome. Alternatively, the molecule may comprise a Smac-6 polypeptide, ubiquitin, and a proteosome. The Smac polypeptide, ubiquitin, and proteosome include any of the species for each respective molecule described herein.

The invention also encompasses a kit. Typically, the kit will include an isolated Smac polypeptide or an isolated Smac wild-type gene or both, an appropriate container and instructions detailing how to properly use the contents of the kit. The kit may be used to detect Smac polypeptides, or as a hybridization kit. Alternatively, the kit may be employed as a source for Smac polypeptide for use in any of the methods described herein. In another embodiment, the kit is employed for detecting a Smac gene comprising PCR primers spanning the Smac gene, and the kit will include a positive control, and sequencing products. In yet another alternative embodiment, the kit may be employed as a standard for use in drug design. For example, the kit may include Smac wild-type polypeptide, isolated c-IAP1 and/or c-IAP2, mammalian ubiquitin, and one or more of E₁, E₂, and E₃ from a suitable mammalian source. The kit may be utilized as a standard to compare the auto-ubiquitylation activity of wild type Smac polypeptide versus the activity of Smac polypeptide fragments or mutant Smac polypeptides being tested as potential apoptotic drugs.

### Definitions

An “antigen” (Ag) is a molecule that can bind specifically to an antibody (Ab). Their name arises from their ability to generate antibodies. Each Ab molecule has a unique Ag binding pocket that enables it to bind specifically to its corresponding antigen. Abs are produced by B cells and plasma cells in response to infection or immunization, bind to and neutralize pathogens, or prepare them for uptake and destruction by phagocytes.

“Auto-ubiquitylation” describes a process wherein a molecule, in whole or in part, causes its own ubiquitylation, such as in the ubiquitylation of a protein.

“Caspase” is defined as a group of cysteine proteases involved in apoptosis.

“Degenerate code” is one in which a variety of symbols or groups of letters code each different word. The genetic code is said to be degenerate because more than one nucleotide triplet codes for the same amino acid.

A “Degenerative variant” is a protein that has substantially the same function as the wild type protein, but has a different amino acid sequence, where the sequence difference results from the degenerate code.

A “gene” is a hereditary unit that has one or more specific effects upon the phenotype of the organism that can mutate to various allelic forms.

A “host organism” is an organism that receives a foreign biological molecule, including an antibody or genetic construct, such as a vector containing a gene.

“Homlogy” describes the degree of similarity in nucleotide or protein sequences between individuals of the same species or among different species. As the term is employed herein, such as when referring to the homology between either two proteins or two nucleotide sequences, homology refers to molecules having substantially the same function, but differing in sequence. Most typically, the two homologous molecules will share substantially the same sequence, particularly in conserved regions, and will have sequence differences in regions of the sequence that does not impact function.

“Mutation” is defined as a phenotypic variant resulting from a changed or new gene.

“Mutant” is an organism bearing a mutant gene that expresses itself in the phenotype of the organism. Mutants include both changes to a nucleic acid sequence, as well as elimination of a sequence or a part of a sequence. In addition polypeptides can be expressed from the mutants.

A “nucleic acid” is a nucleotide polymer better known as one of the monomeric units from which DNA or RNA polymers are constructed, it consists of a purine or pyrimidine base, a pentose, and a phosphoric acid group.

“Peptide” is defined as a compound formed of two or more amino acids, with an amino acid defined according to standard definitions, such as is found in the book “A Dictionary of Genetics” by King and Stansfield.

“Plasmids” are double-stranded, closed DNA molecules ranging in size from 1 to 200 kilo-bases. Plasmids are incorporated into vectors for transfecting a host with a nucleic acid molecule.

A “polypeptide” is a polymer made up of less than 350 amino acids.

“Protein” is defined as a molecule composed of one or more polypeptide chains, each composed of a linear chain of amino acids covalently linked by peptide bonds. Most proteins have a mass between 10 and 100 kilodaltons. A protein is often symbolized by its mass in kDa.

“Smac” stands for the second mitochondria-derived activator of caspase after cytochrome c.

A “vector” is a self-replication DNA molecule that transfers a DNA segment to a host cell.

“Ubiquitin” herein is meant a polypeptide that is ligated to another polypeptide by ubiquitin ligase enzymes. The ubiquitin can be from any species of organism, preferably a eukaryotic species. Preferably, the ubiquitin is mammalian. More preferably, the ubiquitin is human ubiquitin.

“Wild-type” is the most frequently observed phenotype, or the one arbitrarily designated as “normal”. Often symbolized by “+” or “WT.”

### EXAMPLES

Examples 1-7 below detail the ability of Smac polypeptide to selectively cause the rapid degradation of c-IAP1 and c-IAP2, but not XIAP and Livin, although it effectively promotes the auto-ubiquitylation of all of them.

In the examples below, where indicated, the following experimental procedures and reagents were employed:

Antibodies and reagents—The polyclonal antibodies that recognize residues 527-546 of human c-IAP1, residues 507-524 of human c-IAP2 and residues 244-263 of human XIAP, and the monoclonal antibody against human ubiquitin were purchased from R&D Systems. HRP-conjugated anti-GST antibody, anti-FLAG M2 antibody, and anti-c-Myc monoclonal antibody were obtained from Sigma. The monoclonal anti-Livin antibody was obtained from IMGENEX and the monoclonal anti-Actin antibody was obtained from Santa Cruz Biotechnology. The polyclonal antiserum against Smac polypeptide was generated by immunizing rabbits with recombinant Smac protein as previously described (7). The mammalian ubiquitin, rabbit ubiquitin activating enzyme (E1) and various recombinant human ubiquitin conjugating enzymes (E2) were obtained from Boston Biochem.

Plasmids—The plasmids for the GST fusion proteins of human c-IAP1, c-IAP2 and XIAP (29), and the C-terminal (His)₉-tagged mature form Smac polypeptide (7) were prepared as previously described. The cDNAs for Livin α and Livin β were individually inserted into the pGEX-4T-2 (Amersham) to generate GST fusion proteins. The p3×FLAG-CMV-7 vector (Sigma) was used for the expression of IAP proteins in mammalian cells. The N-terminal ubiquitin-fused and C-terminal c-Myc-tagged Smac wild type (WT) polypeptide and the Ala deletion mutant polypeptide (ΔA) were generated by PCR and subcloned into the pcDNA3.1 (−) mammalian expression vector (Invitrogen) according to the reported technique (34, 35). The mutation of the conserved His residue to Ala in the RING domain of respective IAPs (c-IAP1H588A, c-IAP2H574A, XIAP H467A, Livin α H269A and Livin β H251A) was made using the QuickChange Multi Site-Directed Mutagenesis Kit (Stratagene). Full length c-IAP1 and XIAP and different c-IAP 1 truncation mutants were generated by PCR and subcloned into the pTYB11 (New England Biolabs). All constructs were confirmed by sequencing.

Protein expression and purification—All of the recombinant proteins were expressed in E. coli strain BL21 (DE3). The GST-fused IAP proteins were purified with Glutathione Sepharose affinity chromatography, followed by Superdex 200 gel-filtration chromatography if necessary. The C-terminal (His)₉-tagged wild type Smac polypeptide and Smac ΔA polypeptide were purified with Ni-NTA Sepharose affinity chromatography. The non-tagged, full length c-IAP1 and XIAP, and the truncated c-IAP1 mutants were purified from the Chitin affinity column after DTT induction according to the manufacture's protocols (New England Biolabs). The protein concentrations were determined by the modified Bradford method (36).

In vitro ubiquitylation assays—In vitro ubiquitylation assays were carried out as previously described (29). IAP proteins (200 nM) were incubated with or without Smac polypeptide for 2 hours at 30° C. in a reaction system containing 50 mM Tris-HCl (pH 7.5), 50 mM NaCl, 2 mM Mg-ATP, 20 μM mammalian ubiquitin, 100 nM rabbit ubiquitin-activating enzyme (E1) and 400 nM of recombinant human ubiquitin-conjugating enzymes (E2). The reactions were stopped by adding equal volumes of 2×SDS sample loading buffer followed by Western Blot analysis.

Transfection of cultured Cells—HeLa cells were grown in DMEM supplemented with 10% fetal bovine serum. Cells were seeded onto 6-well plates the day before transfection and transfected at 70% confluence by using Lipofectin combined with Plus reagent (Invitrogen) according to the manufacture's protocols. The IAP expression plasmids (3 μg each for c-IAP1, c-IAP2 and XIAP, and 250 ng each for Livin αand Livin β) together with 2.5 μg of Smac expression plasmids or pcDNA3.1(−) blank plasmid were used for single-well transfection in the absence of antibiotics. The cells were harvested 12 hours after the transfection and lysed with 0.5% CHAPS in 20 mM HEPES (pH 7.4), 10 mM KCl, 1 mM MgCl₂ and 1 mM DTT. The lysates were centrifuged and the protein concentrations of the supernatants were quantified by the modified Bradford method (36).

Western Blot analysis—Proteins were resolved by SDS-PAGE and transferred to nitrocellulose filters. The filters were blocked with 5% milk and probed with antibodies as indicated. The signals were visualized with the enhanced chemiluminescence method.

### Example 1

### Smac Prevents the Accumulation of Both c-IAP1 and c-IAP2 in HeLa Cells

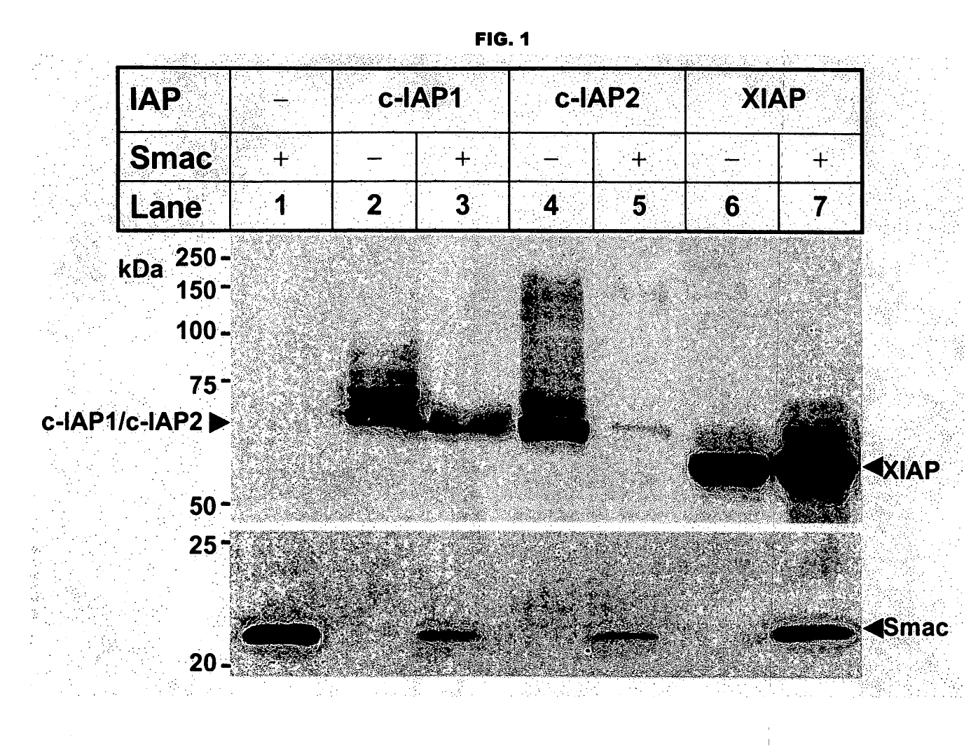

To investigate the effect of Smac polypeptide on IAP protein levels, HeLa cells were transfected with the expression vectors for Smac and various IAPs. The results are shown in FIG. 1. Briefly, HeLa cells were either transfected with IAP alone (Lane 2, 4 and 6), or together with Smac polypeptide (Lane 3, 5 and 7), or with Smac polypeptide alone (Lane 1). Equal amounts of cell lysates (7 μg) were immunoblotted with either the HRP-conjugated anti-FLAG antibody (shown in the top panel) in order to detect N-terminal 3×FLAG tagged IAPs, or with the anti-c-Myc antibody (bottom panel) to detect C-terminal c-Myc tagged Smac. The ubiquitin fusion technique was used to secure the production of mature Smac protein with the expected N-terminal AVPI motif (6, 37).

The HeLa cells transiently transfected with c-IAP1 or c-IAP2 expression vector produced not only the expected full length c-IAP1 or c-IAP2, but also the multiple higher molecular mass forms that are characteristic of ubiquitylated products (as shown in lanes 2 and 4 of FIG. 1). This was consistent with previous reports that both c-IAP1 and c-IAP2 are ubiquitin-protein ligases (E3) and are capable of directing auto-ubiquitylation (16, 22). In contrast, the transiently expressed XIAP, under the same conditions, showed only one or two bands above the major band of full length XIAP (as shown in lane 6 of FIG. 1). The protein levels of both c-IAP1 and c-IAP2, but not XIAP, were significantly decreased when the mature form of Smac was co-expressed (as shown in lanes 2, 4 and 7 of FIG. 1).

It is known that the Drosophila functional homologs of Smac, Reaper, Hid and Grim can promote the degradation of mammalian IAP proteins bearing normal E₃ activity (23, 26). Two of these Smac homologs, Grim and Reaper, also reduce the levels of IAPs by suppressing global protein synthesis (23, 26). In the present work, it was unlikely that the decrease of c-IAP1 and c-IAP2 was caused by the Smac-mediated inhibition of protein synthesis since XIAP under similar conditions was unaffected.

### Example-2

### Smac Promotes the Degradation of c-IAP1 and c-IAP2 by Enhancing Their Auto-Ubiquitylation in HeLa Cells

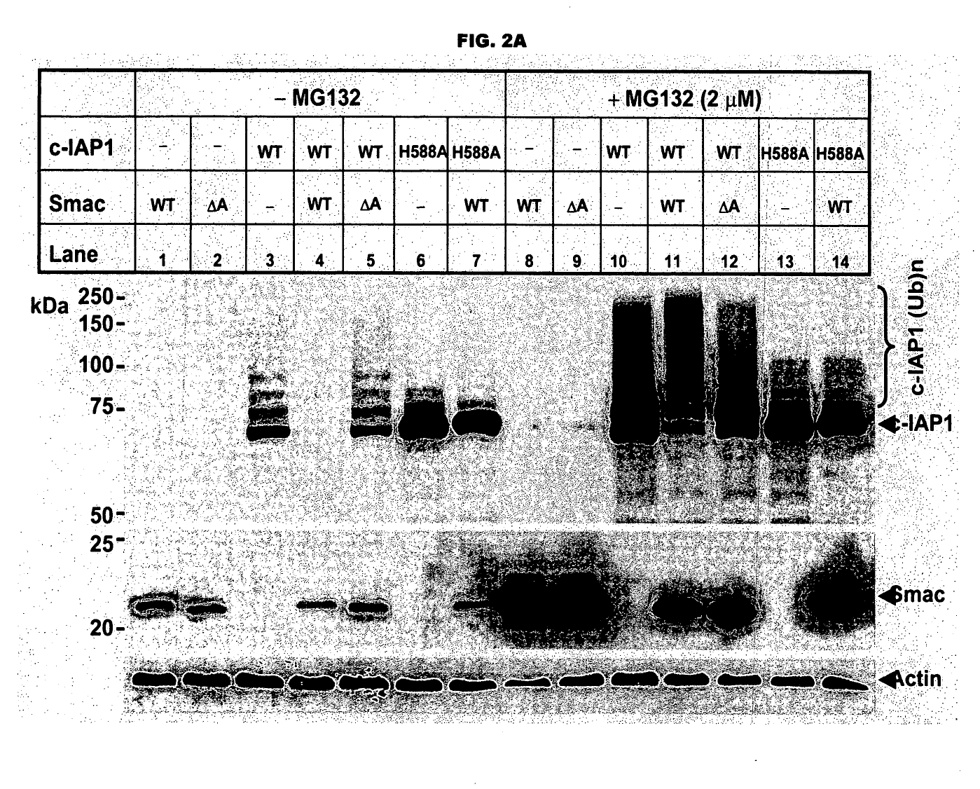

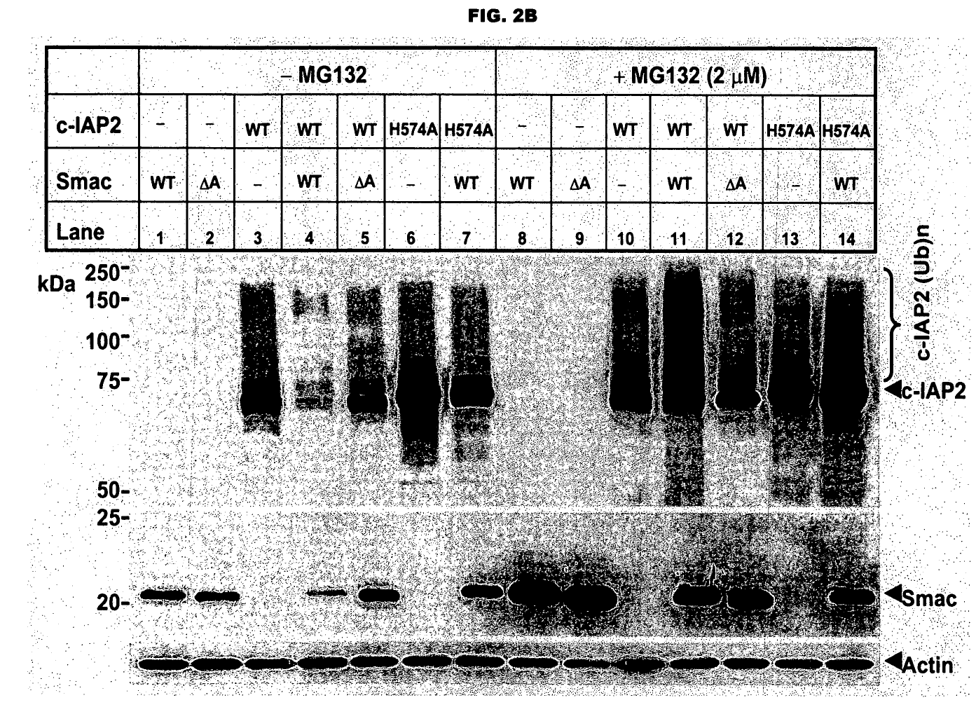

Because it is known that the His⁵⁸⁸ residue in the RING finger domain is important for the IAP E₃ activity (22), this residue was mutated to test whether Smac stimulated the ubiquitylation-mediated degradation of both c-IAP1 and c-IAP2. In each case, His⁵⁸⁸ in c-IAP1 and His⁵⁷⁴ in c-IAP2 was mutated to Ala (H588A mutant and H574A mutant, respectively) and the results are depicted in FIGS. 2A and 2B. The c-IAP1 WT or H588A mutant plasmids together with either Smac WT or Smac ΔA mutant, or with pcDNA3.1 (−) blank plasmid were co-transfected into HeLa cells (FIG. 2A). The c-IAP2 WT or H574A mutant plasmids together with either Smac WT or Smac ΔA mutant, or with pcDNA3.1 (−) blank plasmid were co-transfected into HeLa cells were co-transfected into HeLa cells (FIG. 2B). As controls, HeLa cells were also transfected with either Smac WT or ΔA plasmids together with the blank p3×FLAG-CMV-7 plasmid. The transfected cells were cultivated either in the absence (see lanes 1-7 of FIGS. 2A and 2B) or in the presence (see lanes 8-14 of FIGS. 2A and 2B) of MG132. Equal amounts of soluble proteins (7 μg) from the transfected cells were immunoblotted with either the HRP-conjugated anti-FLAG antibody (see top panel of FIGS. 2A and 2B) to detect N-terminal 3×FLAG tagged IAPs, or with the anti-c-Myc antibody (see middle panel of FIGS. 2A and 2B) to detect C-terminal c-Myc tagged Smac. The anti-Actin immunoblotting results were to show equal protein loading (see bottom panel of FIGS. 2A and 2B).

The H588A mutant c-IAP1 exhibited a much higher expression level than the wild type in HeLa cells (see, for example, a comparison of lanes 3 and 6 of FIG. 2A). The amount of wild type c-IAP1 was also dramatically increased by the addition of proteasome inhibitor MG132 to the culture medium (see lane 10, FIG. 2A). MG132, however, did not cause a significant increase in the H588A c-IAP1 protein level (see lane 13, FIG. 2A). These observations were in agreement with the previous reports that c-IAP1 is continuously down-regulated by ubiquitylation-dependent degradation and such ubiquitylation is mediated by the E₃ activity of c-IAP1 itself (22).

In the presence of MG132, co-expression of wild type Smac led to a reduction in the level of non-ubiquitylated and mono-/di-ubiquitylated wild type c-IAP1 and a concomitant increase in the level of highly ubiquitylated c-IAP1 forms (see lane 11 of FIG. 2A). In the absence of MG132, co-expression of Smac resulted in nearly complete disappearance of c-IAP1 (see lane 4 of FIG. 2A). This data illustrates that Smac promoted the poly-ubiquitylation and degradation of c-IAP1.

It is known that the processed Smac and other IAP antagonists have a conserved N-terminal IAP-binding motif (IBM) with an initial Ala residue. Mutation of this Ala or deletion of the IBM abolishes the specific binding of these proteins to IAP (38, 39). To assess whether Smac enhancement of c-IAP1 ubiquitylation and degradation requires the same binding, c-IAP1 was co-transfected with a Smac mutant lacking the initial Ala (Smac ΔA) that was also produced using the ubiquitin fusion expression vector. As expected, Smac ΔA did not enhance the auto-ubiquitylation of c-IAP1 (see a comparison of lanes 5 and 3, and lanes 13 and 10 of FIG. 2A), suggesting that the specific binding of Smac to c-IAP1 via its IBM is required for Smac to enhance the auto-ubiquitylation of c-IAP1. In agreement with the above observations, Smac did not cause a drastic change in the level of E₃-negative H588A c-IAP1, either in the absence or in the presence of MG132 (see lanes 6 and 7, and lanes 13 and 14 of FIG. 2A, respectively). Smac likewise strongly promoted the ubiquitylation and degradation of c-IAP2 (see FIG. 2B). Mutation of His⁵⁷⁴ to Ala in c-IAP2 also significantly increased the expression level of c-IAP2 (see FIG. 2B).

As mentioned above, the level of c-IAP1 was significantly elevated by the addition of MG132. In contrast, c-IAP2 did not show such a significant change when MG132 was added (for example, compare lanes 3 and 10 in FIG. 2B). Such a difference made it reasonable to speculate that in the absence of Smac, c-IAP2's E₃ was much less active than c-IAP1 in directing its auto-ubiquitylation.

It should be noted that although His⁵⁸⁸ in c-IAP1 and His⁵⁷⁴ in c-IAP2 are critical for their E₃ activities, mutation of this residue to Ala greatly reduced, but did not completely abolish their E₃ activity, as revealed by the background ubiquitylated product ladders (see lanes 6, 7, 13 and 14 of FIGS. 2A and 2B). This residual activity was also observed in an in vitro ubiquitylation assay using purified mutant c-IAP proteins (data not shown).

### Example 3

### Smac Does Not Promote the Degradation of XIAP and Livin in HeLa Cells

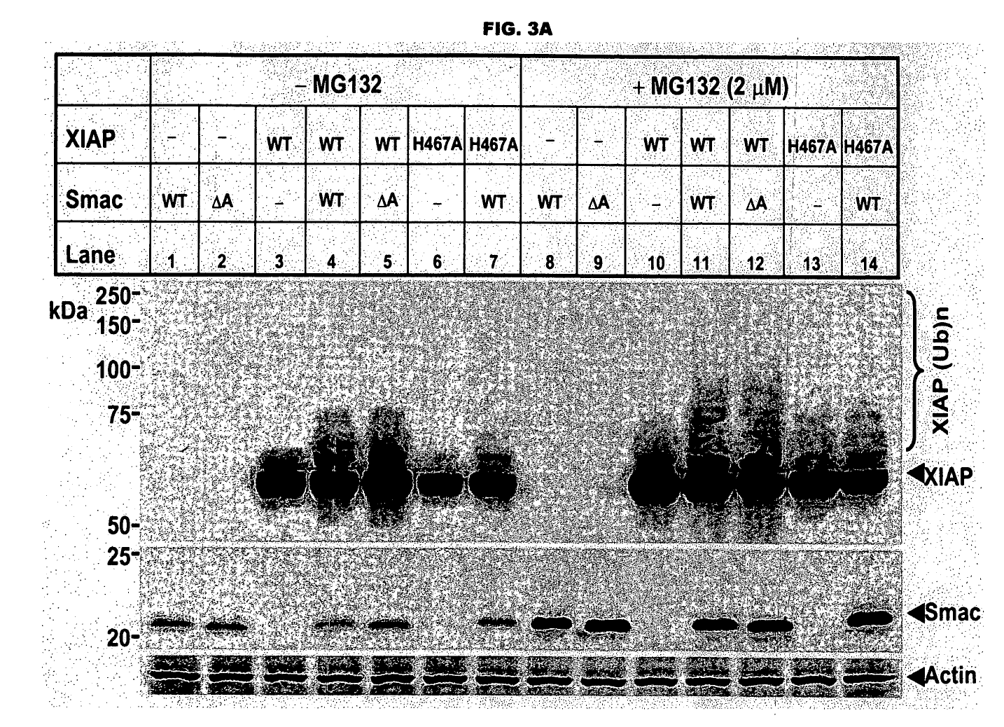

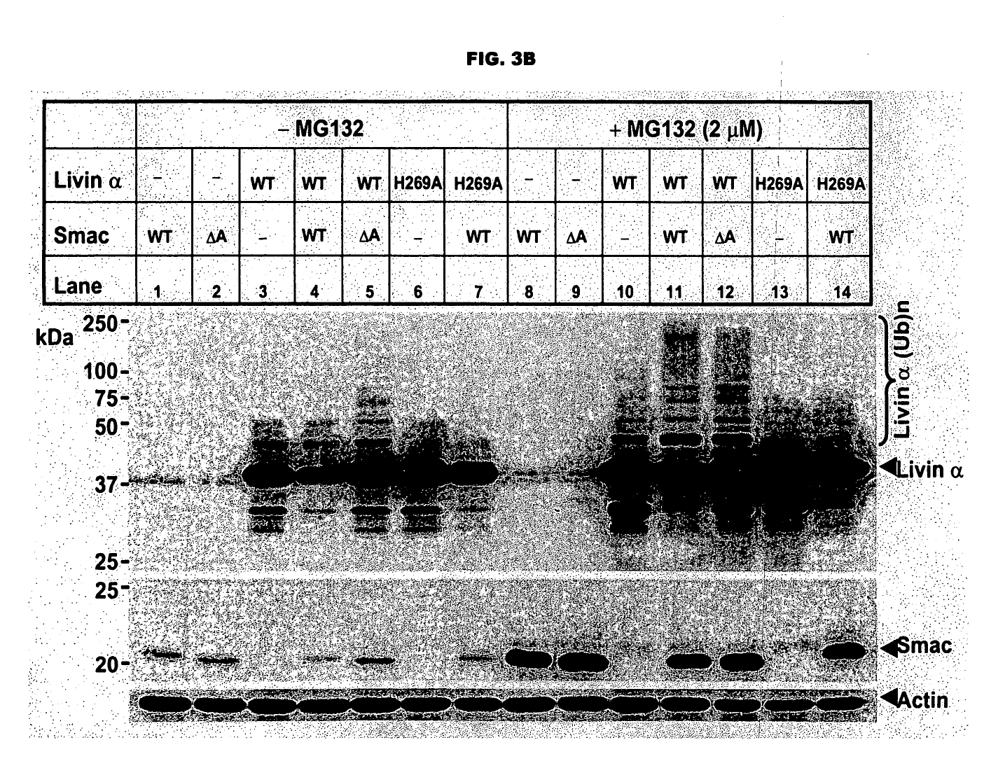

It is known that XIAP is ubiquitylated by its own E₃ activity when expressed in HEK 293T cells and that this auto-ubiquitylation and subsequent degradation is stimulated by Reaper (26). In the current study, XIAP transiently expressed in HeLa cells was ubiquitylated as well; however, mono-ubiquitylation rather than poly-ubiquitylation of XIAP was the major product (see FIG. 3A). Briefly, XIAP WT or H467A mutant plasmids together with either Smac WT or ΔA, or with pcDNA3.1(−) blank plasmid, were co-transfected into the HeLa cells. As controls, HeLa cells were also transfected with either Smac WT or ΔA plasmids together with the blank p3×FLAG-CMV-7 plasmid. The transfected cells were cultivated either in the absence (see lanes 1-7 of FIG. 3A) or in the presence (see lanes 8-14 of FIG. 3A) of MG132. Equal amounts of soluble proteins (7 μg) from the transfected cells were immunoblotted with either the HRP-conjugated anti-FLAG antibody (see top panel of FIG. 3A) to detect N-terminal 3×FLAG tagged IAPs, or with the anti-c-Myc antibody (see middle panel of FIG. 3A) to detect Smac. The anti-Actin immunoblotting results were to show equal protein loading (see bottom panel of FIG. 3A).

Similar to c-IAP1 and c-IAP2, XIAP ubiquitylation also depended on its E₃ activity, for the H467A E₃-negative mutant gave either no band or a much weaker mono-ubiquitylation band (see lanes 3, 5, 8 and 10 of FIG. 3A). Although more poly-ubiquitylated XIAP products could be detected in the presence of MG132, they were much less significant compared to that of c-IAP1 and c-IAP2, as detailed in Example 2.

Unlike that of c-IAP1 and c-IAP2, the overall amount of XIAP was not significantly affected by the co-transfected Smac, although Smac still promoted XIAP ubiquitylation. In the absence of MG132, co-expression of Smac only had a negligible effect on the level of XIAP (see lane 4 of FIG. 3A), whereas under similar conditions Smac reduced both c-IAP1 and c-IAP2 to a nearly non-detectable level (see FIGS. 2A and 2B). This result was consistent with the report by Silke et al. that Smac does not promote XIAP degradation (27).

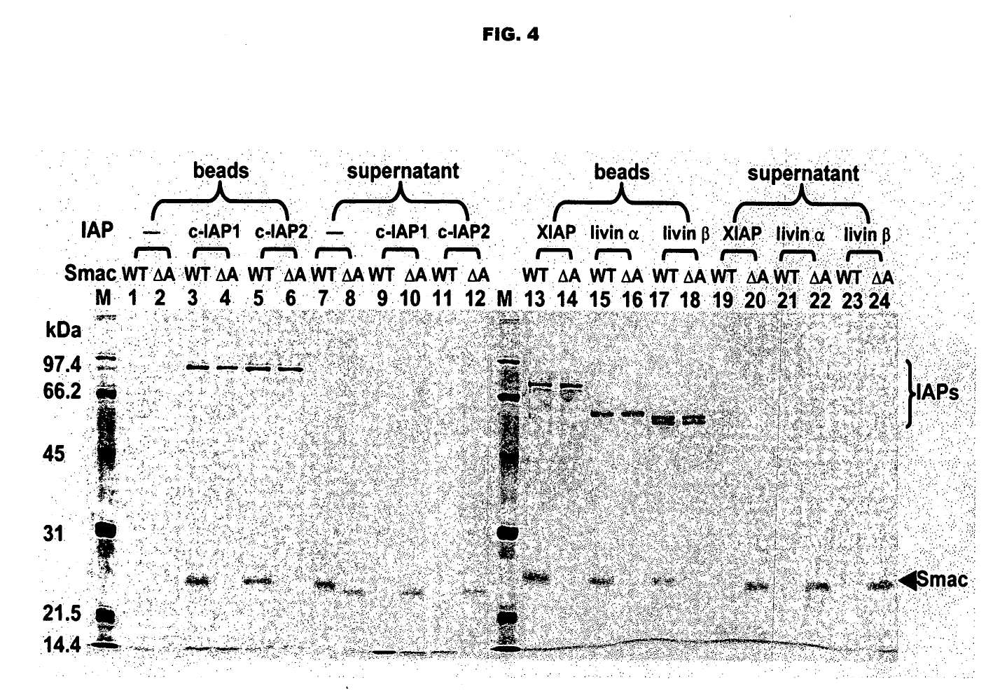

The ability of various IAP proteins to bind Smac WT polypeptide and Smac ΔA mutant polypeptide was also examined. The results are depicted in FIG. 4. Briefly, purified IAP proteins (GST fusion form; as indicated in FIG. 4) were incubated with equimolar concentrations of either Smac or Smac ΔA in a buffer containing 20 mM phosphate (pH 7.4), 200 mM NaCl and 0.05% Tween 20 for 2 hours at 4° C. The GST-fused IAP proteins were pulled down by Glutathione Sepharose beads. The resulting supernatants were subjected to SDS-PAGE followed by silver staining. Lane M of FIG. 4 is the protein marker. Unexpectedly, as detailed in FIG. 4, the Smac ΔA mutant, despite its failure to bind to various IAPs, also stimulated the auto-ubiquitylation of XIAP and Livin (see lanes 4 and 5 and 11 and 12 in FIG. 3). The reason for this IBM-independent acceleration on XIAP and Livin ubiquitylation is unknown.

In the current study, Livin was also transiently expressed in HeLa cells to determine the ubiquitylation activity (see FIG. 3B). Briefly, Livin α WT or H269A mutant plasmids together with Smac WT or Smac ΔA mutant, or with pcDNA3.1(−) blank plasmid, were co-transfected into HeLa cells. As controls, HeLa cells were also transfected with either Smac WT or ΔA plasmids together with the blank p3×FLAG-CMV-7 plasmid. The transfected cells were cultivated either in the absence (see lanes 1-7 of FIG. 3B) or in the presence (see lane 8-14 of FIG. 3B) of MG132. Equal amounts of soluble proteins (7 μg) from the transfected cells were immunoblotted with either the HRP-conjugated anti-FLAG antibody (see top panel of FIG. 3B) to detect N-terminal 3×FLAG tagged IAPs, or with the anti-c-Myc antibody (see middle panel of FIG. 3B) to detect Smac. The anti-Actin immunoblotting results were to show equal protein loading (see bottom panel of FIG. 3B). Accordingly, Smac effectively stimulates auto-ubiquitylation of Livin α, which slightly promoted the degradation of Livin α (see lane 4 of FIG. 3B). Livin β, an alternatively spliced form of Livin, lacks 18 residues between the BIR and the RING domains compared to Livin α (40,41). The transfection results of Livin β were substantially similar to that of Livin α (data not shown).

In contrast to c-IAP1 and c-IAP2, the mutation of the corresponding His residue in the RING finger domain of XIAP and Livin did not considerably enhance their expression, although the mutation did greatly reduce their E₃ activity. Taken together, these observations suggest that the levels of XIAP and Livin are not substantially regulated by auto-ubiquitylation/proteasomal degradation in HeLa cells.

### Example-4

### Different IAP Proteins Require the Same Ubiquitin-Conjugating Enzymes In Vitro for their E₃ Activity

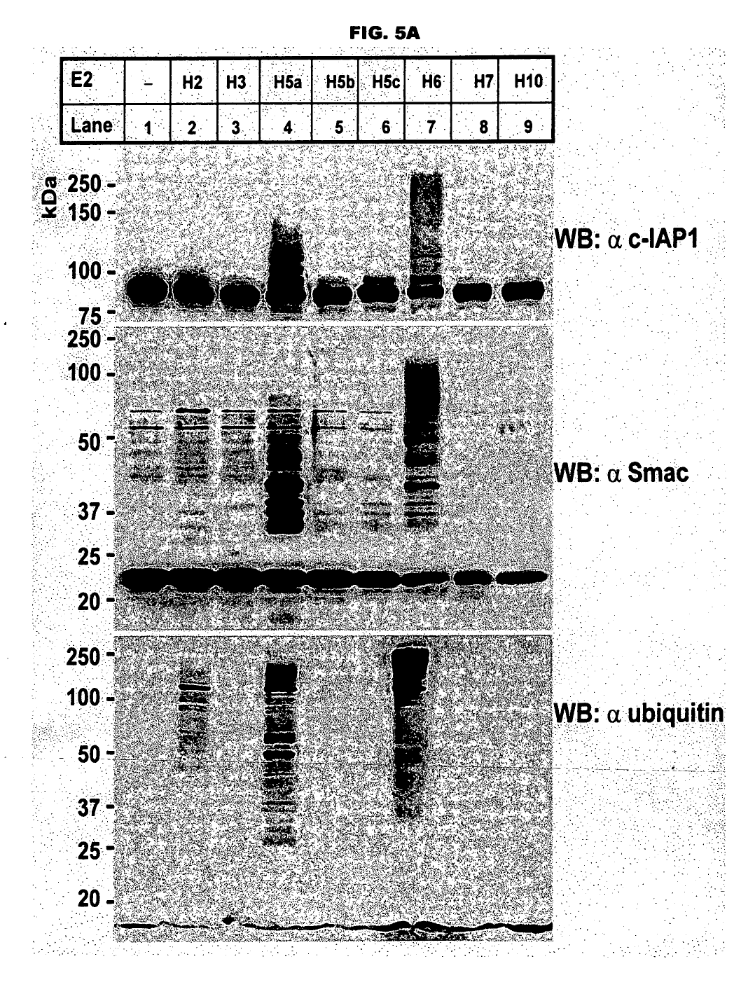

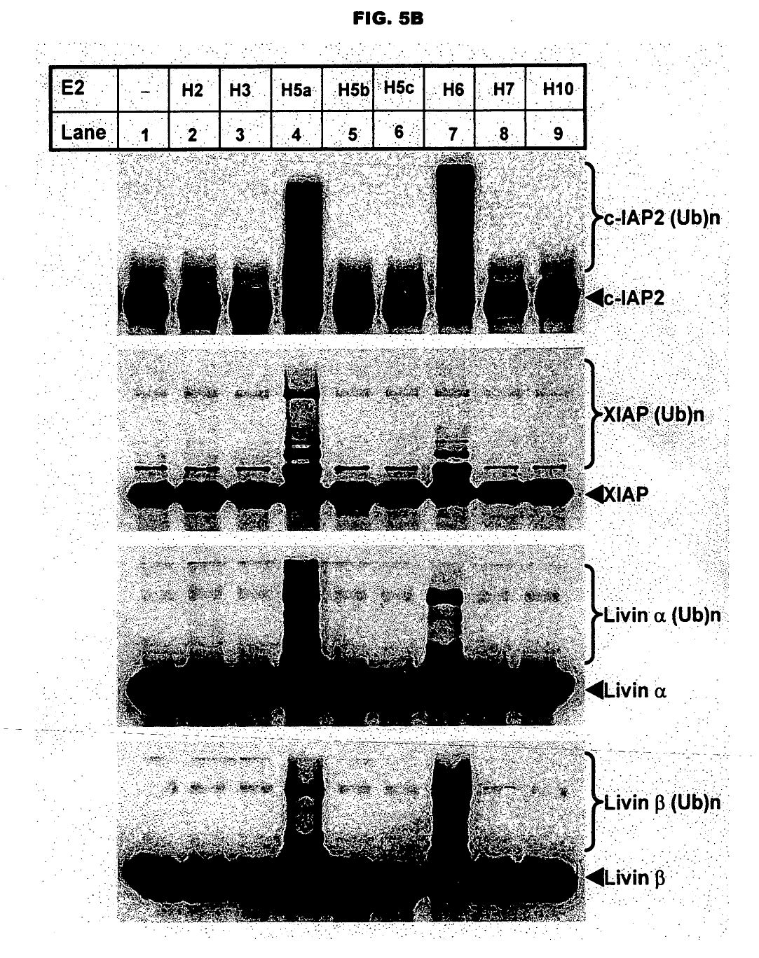

To compare the E₃ activities of human IAPs, an in vitro ubiquitylation assay was performed (see FIGS. 5A and 5B). The assay system contained purified ubiquitin, ubiquitin-activating enzyme (E1) and ubiquitin-conjugating enzyme (E2) and was employed to screen a panel of ubiquitin-conjugating enzymes available from Boston Biochem. Briefly, the purified IAP proteins (200 nM) were incubated with Smac (400 nM) for 2 hours at 30° C. in a reconstituted assay system consisting of 50 mM Tris-HCl (pH 7.5), 50 mM NaCl, 2 mM Mg-ATP, 20 μM mammalian ubiquitin, 100 nM rabbit ubiquitin-activating enzyme (E1) and 400 nM of different recombinant human ubiquitin-conjugating enzymes (E2). The reactions were stopped by adding equal volumes of 2×SDS sample loading buffer and the products were subjected to SDS-PAGE followed by immunoblotting with different antibodies. The ubiquitylation reaction products for c-IAP1 (GST fusion form) and Smac were immunoblotted with anti-c-IAP1 antibody (see top panel of FIG. 5A) and anti-Smac antibody (see middle panel of FIG. 5A), respectively. The filter for c-IAP1 detection was stripped and re-probed with anti-ubiquitin antibody (see bottom panel of FIG. 5A). The ubiquitylation reaction products for c-IAP2 (GST fusion form), XIAP (natural form, without tag) and Livin α and β (GST fusion form) were immunoblotted with the respective antibodies against each IAP protein (see FIG. 5B). All of these IAP proteins exhibited strong E₃ activity when UbCH5a and UbCH6 were used as the E2 as manifested by the characteristic poly-ubiquitylation ladders (see lanes 4 and 7 of FIG. 5).

### Example 5

### Smac does not Promote Auto-Ubiquitylation of XIAP In Vitro

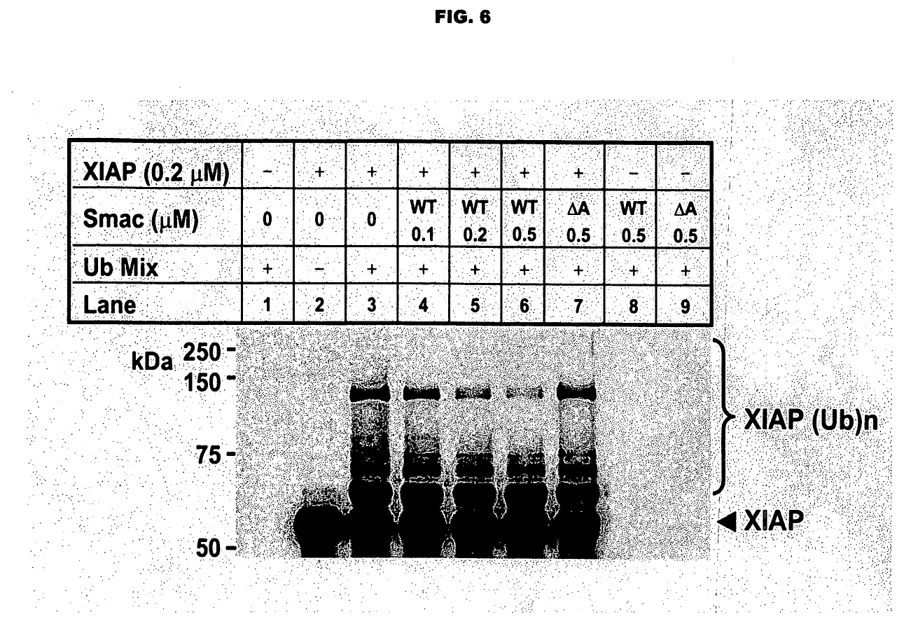

To determine whether Smac promotes auto-ubiquitylation of XIAP, purified XIAP (without tag) was incubated for 2 hours at 30° C. either with or without different concentrations of Smac or with Smac ΔA in the reconstituted ubiquitylation reaction system (Ub Mix) consisting of 50 mM Tris-HCl (pH 7.5), 50 mM NaCl, 2 mM Mg-ATP, 20 μM mammalian ubiquitin, 100 nM rabbit ubiquitin-activating enzyme (E1) and 400 nM UbCH6 (E2). The reactions were stopped by adding equal volumes of 2×SDS sample loading buffer and the products were subjected to SDS-PAGE followed by immunoblotting with anti-XIAP antibody. The purified XIAP was active in directing poly-ubiquitylation. But the auto-ubiquitylation of XIAP was not enhanced by Smac. The amount of ubiquitylated forms of XIAP with a high molecular weight of ˜150 kDa was even slightly reduced by Smac (see FIG. 6).

### Example 6

### Smac Promotes Auto-Ubiquitylation of c-IAP1 In Vitro Through Specific Association with the BIR Domains

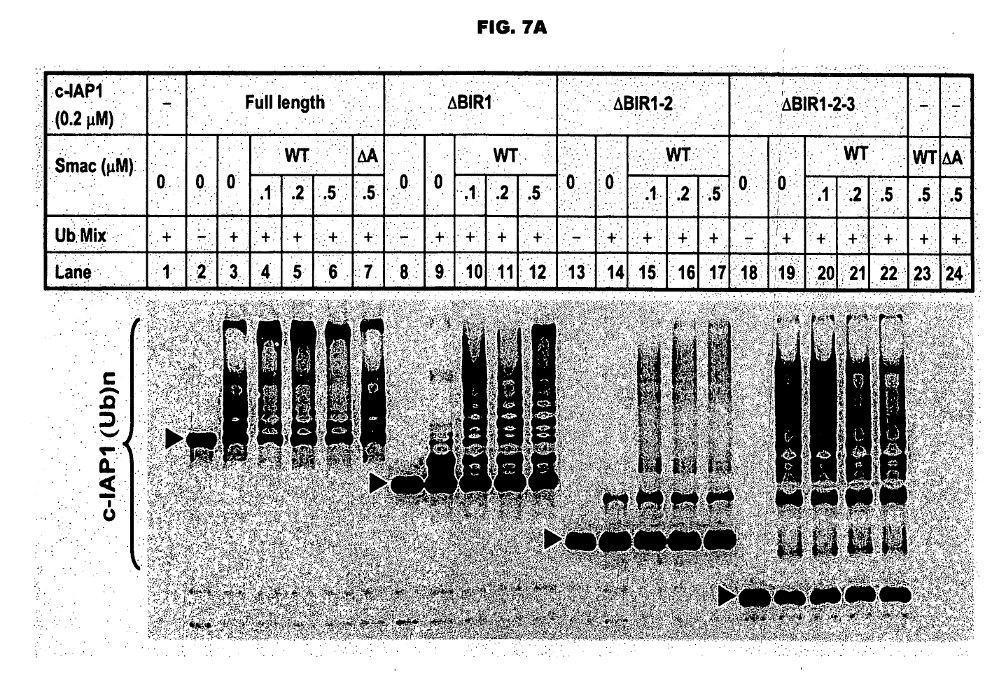

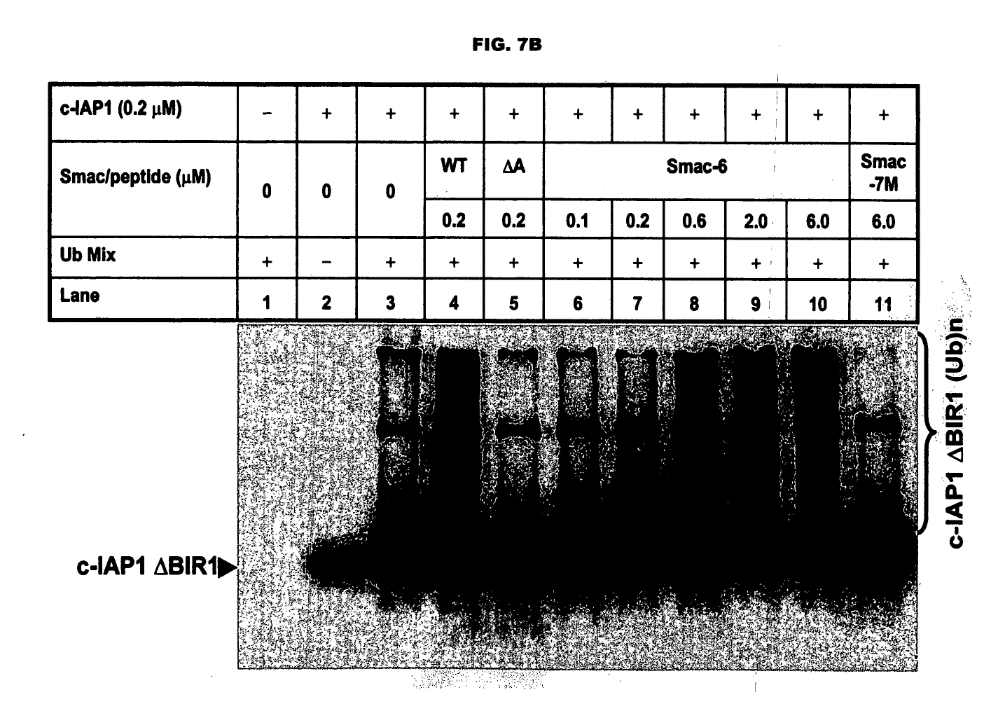

In the reconstituted ubiquitylation reaction system (Ub Mix) described in Example 5, the purified c-IAP1 proteins were incubated for 2 hours at 30° C. either without or with various concentrations of Smac, as shown in FIG. 7A, or c-IAP1 ΔBIR1 were incubated with various concentrations of Smac N-terminal peptide Smac-6, as shown in FIG. 7B. Smac ΔA and the peptide Smac-7M were used as negative controls. The reactions were stopped by adding equal volumes of 2×SDS sample loading buffer and the products were subjected to SDS-PAGE followed by immunoblotting with anti-c-IAP1 antibody. The arrows indicate the unmodified full length or deleted c-IAP1.

The observation that Smac promotes c-IAP1 auto-ubiquitylation in HeLa cells was further confirmed in vitro by using UbCH6 as the E2. Smac, at equimolar concentration to c-IAP1, significantly enhanced c-IAP1 auto-ubiquitylation (see lane 5 of FIG. 7A). Consistent with the transfection result, SmacΔA failed to promote such auto-ubiquitylation (see lane 7 of FIG. 7A). Similar results were also obtained with Smac and c-IAP2 (data not shown).

The Smac mutant lacking the initial Ala residue did not bind IAP proteins, and consequently did not promote the E₃ activity of either c-IAP1 or c-IAP2 in HeLa cells or in vitro. These results demonstrate that the interaction between the IBM of Smac and the BIR domains of IAP was essential and that the BIR domains function, in part, to regulate the E₃ activity of the RING finger. To verify this, stepwise deletion was performed of the first BIR (ΔBIR1, residues 1-161), the first two BIRs (ΔBIR1-2, residues 1-265), or all the three BIR domains (ΔBIR1-2-3, residues 1-339) from c-IAP1. All these mutants and the full-length wild type c-IAP1 were purified as a non-tagged form by using the pTYB11 Intein system (New England Biolabs).

The c-IAP1 mutants with one, two or all three BIR domains deleted were still active in directing their auto-ubiquitylation (see FIG. 7A). The basal activity of ΔBIR1 and ΔBIR1-2 was very low, but was significantly enhanced by Smac, whereas the basal activity of ΔBIR1-2-3 was comparably high and could no longer be enhanced by Smac. This suggested that the second and the third BIR domains of c-IAP1 strongly inhibit the E₃ activity of the C-terminal RING.

### Example 7

### Smac N-Terminal Hexapeptide is Sufficient to Stimulate the Ubiquitin-Protein Isopeptide Ligase Activity of c-IAP1

It is known that the N-terminal peptides of Smac can mimic Smac protein in removing XIAP's inhibition on caspase-3 activation (38, 39). To determine whether the binding of Smac N-terminal peptide to the BIR domains of c-IAP1 was sufficient to promote its ubiquitin-protein isopeptide ligase activity, Smac hexapeptide activity was examined in vitro. The Smac N-terminal hexapeptide (Smac-6, which is SEQ ID NO 2) effectively stimulated E₃ activity of c-IAP1ΔBIR1 (see FIG. 7B). As controls, both Smac ΔA and the peptide with an extra Met before the AVPI motif (Smac-7M) failed to activate c-IAP1.

Accordingly, there has been shown and described a method and a composition that may be employed to selectively promote the autodegradation of c-IAP. It is apparent to those skilled in the art, however, that many changes, variation, modifications, and other uses and applications to the method for promoting c-IAP autodegradation are possible. Moreover, these changes, variations, modifications, and other uses do not depart from the spirit and scope of the invention.

### REFERENCES

1. Liston, P., Fong, W. G., and Korneluk, R. G. (2003) Oncogene 22, 8568-8580
2. Wang, S. L., Hawkins, C. J., Yoo, S. J., Muller, H. A., and Hay, B. A. (1999) Cell 98, 453-463
3. Srinivasula, S. M., Datta, P., Kobayashi, M., Wu, J. W., Fujioka, M., Hegde, R., Zhang, Z., Mukattash, R., Fernandes-Alnemri, T., Shi, Y., Jaynes, J. B., and Alnemri, E. S. (2002) Curr Biol 12, 125-130
4. Wing, J. P., Karres, J. S., Ogdahl, J. L., Zhou, L., Schwartz, L. M., and Nambu, J. R. (2002) Curr Biol 12, 131-135
5. Christich, A., Kauppila, S., Chen, P., Sogame, N., Ho, S. I., and Abrams, J. M. (2002) Curr Biol 12, 137-140
6. Tenev, T., Zachariou, A., Wilson, R., Paul, A., and Meier, P. (2002) Embo J21, 5118-5129
7. Du, C., Fang, M., Li, Y., Li, L., and Wang, X. (2000) Cell 102, 33-42
8. Verhagen, A. M., Ekert, P. G., Pakusch, M., Silke, J., Connolly, L. M., Reid, G. E., Moritz, R. L., Simpson, R. J., and Vaux, D. L. (2000) Cell 102, 43-53
9. Suzuki, Y., Imai, Y., Nakayama, H., Takahashi, K., Takio, K., and Takahashi, R. (2001) Mol Cell 8, 613-621
10. Hegde, R., Srinivasula, S. M., Zhang, Z., Wassell, R., Mukattash, R., Cilenti, L., DuBois, G., Lazebnik, Y., Zervos, A. S., Fernandes-Alnemri, T., and Alnemri, E. S. (2002) J Biol Chem 277, 432-438
11. Martins, L. M., laccarino, I., Tenev, T., Gschmeissner, S., Totty, N. F., Lemoine, N. R., Savopoulos, J., Gray, C. W., Creasy, C. L., Dingwall, C., and Downward, J. (2002) J Biol Chem 277, 439-444
12. Verhagen, A. M., Silke, J., Ekert, P. G., Pakusch, M., Kaufmann, H., Connolly, L. M., Day, C. L., Tikoo, A., Burke, R., Wrobel, C., Moritz, R. L., Simpson, R. J., and Vaux, D. L. (2002) J Biol Chem 277, 445-454
13. van Loo, G., van Gurp, M., Depuydt, B., Srinivasula, S. M., Rodriguez, I., Alnemri, E. S., Gevaert, K., Vandekerckhove, J., Declercq, W., and Vandenabeele, P. (2002) Cell Death Differ 9, 20-26
14. Srinivasula, S. M., Hegde, R., Saleh, A., Datta, P., Shiozaki, E., Chai, J., Lee, R. A., Robbins, P. D., Fernandes-Alnemri, T., Shi, Y., and Alnemri, E. S. (2001) Nature 410, 112-116
15. Yang, Y., and Yu, X. (2003) Faseb J 17, 790-799
16. Huang, H., Joazeiro, C. A., Bonfoco, E., Kamada, S., Leverson, J. D., and Hunter, T. (2000) J Biol Chem 275, 26661-26664
17. Wilson, R., Goyal, L., Ditzel, M., Zachariou, A., Baker, D. A., Agapite, J., Steller, H., and Meier, P. (2002) Nat Cell Biol 4, 445-450
18. Muro, I., Hay, B. A., and Clem, R. J. (2002) J Biol Chem 277, 49644-49650
19. MacFarlane, M., Merrison, W., Bratton, S. B., and Cohen, G. M. (2002) J Biol Chem 277, 36611-36616
20. Hu, S., and Yang, X. (2003) J Biol Chem 278, 10055-10060
21. Olson, M. R., Holley, C. L., Yoo, S. J., Huh, J. R., Hay, B. A., and Kombluth, S. (2003) J Biol Chem 278, 4028-4034
22. Yang, Y., Fang, S., Jensen, J. P., Weissman, A. M., and Ashwell, J. D. (2000) Science 288, 874-877
23. Yoo, S. J., Huh, J. R., Muro, I., Yu, H., Wang, L., Wang, S. L., Feldman, R. M., Clem, R. J., Muller, H. A., and Hay, B. A. (2002) Nat Cell Biol 4, 416-424
24. Hays, R., Wickline, L., and Cagan, R. (2002) Nat Cell Biol 4, 425-431
25. Ryoo, H. D., Bergmann, A., Gonen, H., Ciechanover, A., and Steller, H. (2002) Nat Cell Biol 4, 432-438
26. Holley, C. L., Olson, M. R., Colon-Ramos, D. A., and Kombluth, S. (2002) Nat Cell Biol 4, 439-444
27. Silke, J. H., Kratina, T., Ekert, P. G., Pakusch, M., and Vaux, D. L. (2003) J Biol Chem
28. Fu, J., Jin, Y., and Arend, L. J. (2003) J Biol Chem 278, 52660-52672
29. Yang, Q. H., Church-Hajduk, R., Ren, J., Newton, M. L., and Du, C. (2003) Genes Dev 17, 1487-1496
30. Srinivasula, S. M., Gupta, S., Datta, P., Zhang, Z., Hegde, R., Cheong, N., Fernandes-Alnemri, T., and Alnemri, E. S. (2003) J Biol Chem 278, 31469-31472
31. Suzuki, Y., Takahashi-Niki, K., Akagi, T., Hashikawa, T., and Takahashi, R. (2004) Cell Death Differ 11, 208-216
32. Salvesen, G. S., and Duckett, C. S. (2002) Nat Rev Mol Cell Biol 3, 401-410
33. Deng, Y., Ren, X., Yang, L., Lin, Y., and Wu, X. (2003) Cell 115, 61-70
34. Varshavsky, A. (2000) Methods Enzymol 327, 578-593
35. Baker, R. T., Smith, S. A., Marano, R., McKee, J., and Board, P. G. (1994) J Biol Chem 269, 25381-25386
36. Zor, T., and Selinger, Z. (1996) Anal Biochem 236, 302-308
37. Hunter, A. M., Kottachchi, D., Lewis, J., Duckett, C. S., Korneluk, R. G., and Liston, P. (2003) J Biol Chem 278, 7494-7499
38. Chai, J., Du, C., Wu, J. W., Kyin, S., Wang, X., and Shi, Y. (2000) Nature 406, 855-862
39. Srinivasula, S. M., Datta, P., Fan, X. J., Femandes-Alnemri, T., Huang, Z., and Alnemri, E. S. (2000) J Biol Chem 275, 36152-36157
40. Vucic, D., Stennicke, H. R., Pisabarro, M. T., Salvesen, G. S., and Dixit, V. M. (2000) Curr Biol 10, 1359-1366
41. Ashhab, Y., Alian, A., Polliack, A., Panet, A., and Ben Yehuda, D. (2001) FEBS Lett 495, 56-60
42. Rothe, M., Pan, M. G., Henzel, W. J., Ayres, T. M., and Goeddel, D. V. (1995) Cell 83, 1243-1252
43. Shu, H. B., Takeuchi, M., and Goeddel, D. V. (1996) Proc Natl Acad Sci USA 93, 13973-13978
44. Wang, C. Y., Mayo, M. W., Komeluk, R. G., Goeddel, D. V., and Baldwin, A. S., Jr. (1998) Science 281, 1680-1683
45. Li, X., Yang, Y., and Ashwell, J. D. (2002) Nature 416, 345-347
46. Fotin-Mleczek, M., Henkler, F., Samel, D., Reichwein, M., Hausser, A., Parmryd, I., Scheurich, P., Schmid, J. A., and Wajant, H. (2002) J Cell Sci 115, 2757-2770
47. Micheau, O., and Tschopp, J. (2003) Cell 114, 181-190
48. Kuai, J., Nickbarg, E., Wooters, J., Qiu, Y., Wang, J., and Lin, L. L. (2003) J Biol Chem 278, 14363-14369
49. Roberts, D. L., Merrison, W., MacFarlane, M., and Cohen, G. M. (2001) J Cell Biol 153, 221-228
50. Roy, N., Deveraux, Q. L., Takahashi, R., Salvesen, G. S., and Reed, J. C. (1997) Embo J 16, 6914-6925
51. Ekert, P. G., Silke, J., and Vaux, D. L. (1999) Cell Death Differ 6, 1081-1086
52. Lotocki, G., Alonso, O. F., Frydel, B., Dietrich, W. D., and Keane, R. W. (2003) J Cereb Blood Flow Metab 23, 1129-1136
53. Pickart, C. M. (2001) Annu Rev Biochem 70, 503-533
54. Boutell, C., Sadis, S., and Everett, R. D. (2002) J Virol 76, 841-850
55. Hagglund, R., Van Sant, C., Lopez, P., and Roizman, B. (2002) Proc Natl Acad Sci USA 99, 631-636
56. Jensen, J. P., Bates, P. W., Yang, M., Vierstra, R. D., and Weissman, A. M. (1995) J Biol Chem 270, 30408-30414
57. Nuber, U., Schwarz, S., Kaiser, P., Schneider, R., and Scheffner, M. (1996) J Biol Chem 271, 2795-2800
58. Fulda, S., Wick, W., Weller, M., and Debatin, K. M. (2002) Nat Med 8, 808-815
59. Amt, C. R., Chiorean, M. V., Heldebrant, M. P., Gores, G. J., and Kaufmann, S. H. (2002) J Biol Chem 277, 44236-44243
60. Guo, F., Nimmanapalli, R., Paranawithana, S., Wittman, S., Griffin, D., Bali, P., O'Bryan, E., Fumero, C., Wang, H. G., and Bhalla, K. (2002) Blood 99, 3419-3426
61. Yang, L., Mashima, T., Sato, S., Mochizuki, M., Sakamoto, H., Yamori, T., Oh-Hara, T., and Tsuruo, T. (2003) Cancer Res 63, 831-837

What is claimed is:

1. A method for promoting auto-degradation of an isolated c-IAP, comprising: contacting in vitro the c-IAP with an effective amount of an isolated Smac polypeptide, wherein upon contact the Smac polypeptide binds to the c-IAP and enhances c-IAP auto-ubiquitylation activity thereby causing the ubiquitylation-mediated, auto-degradation of the c-IAP.

2. The method of claim 1, wherein the method further comprises adding an amount of ubiquitin enzyme.

3. The method of claim 1, wherein the Smac polypeptide is selected from the group consisting of
(a) isolated SEQ ID NO 1; and,
(b) homologues of the Smac polypeptide.

4. The method of claim 1, wherein the Smac polypeptide is expressed by an isolated nucleotide sequence selected from the group consisting of:
(a) isolated SEQ ID NO 3; and
(b) homologues, fragments, degenerate variants, and orthologs of the Smac nucleotide sequence.

5. The method of claim 1, wherein the c-IAP is selected from the group consisting of c-IAP 1 and c-IAP2.

6. The method of claim 1, wherein the c-IAP auto-ubiquitylation activity results from the E₃ activity of the c-IAP ring domain.

7. The method of claim 1, wherein the molar ratio of c-IAP to Smac polypeptide is from about 1 to about 1.

8. The method of claim 1, wherein the Smac polypeptide binds to a BIR domain of the c-IAP through its N-terminal IAP-binding motif.

9. The method of claim 8, wherein the N-terminal IAP-binding motif comprises a polypeptide of SEQ ID NO 2.

10. A method for promoting the selective auto-degradation of a c-IAP in a medium having a plurality of IAP molecules, the method comprising and contacting in vitro, the medium with an effective amount of an isolated Smac polypeptide, wherein upon contact the Smac polypeptide binds to the c-IAP in the medium and enhances c-IAP auto-ubiquitylation activity thereby causing the ubiquitylation-mediated, auto-degradation of the c-IAP.

11. The method of claim 10, wherein the Smac polypeptide consists of SEQ ID NO 1.

12. The method of claim 10, wherein the Smac polypeptide is encoded by a nucleotide sequence consisting of SEQ ID NO 3.

13. The method of claim 10, wherein the IAP molecule is a mammalian IAP.

14. The method of claim 10, wherein the IAP molecule is selected from the group consisting of c-IAP1, c-IAP2, XIAP and Livin.

15. The method of claim 10, wherein the c-IAP auto-ubiquitylation activity results from the E₃ activity of the c-IAP ring domain.

16. The method of claim 10, wherein the molar ratio of c-IAP to Smac polypeptide is from about 1 to about 1.

17. The method of claim 10, wherein the Smac polypeptide binds to a BIR domain of the c-IAP through its N-terminal IAP-binding motif.

18. The method of claim 17, wherein the N-terminal IAP-binding motif is a polypeptide having the sequence of SEQ ID NO 2.

19. A method for promoting the auto-degradation of a c-IAP in a cell, the method comprising introducing into the cell an effective amount of an isolated molecule selected from the group consisting of:
(a) a nucleotide sequence encoding a Smac polypeptide under conditions resulting in the expression of the Smac polypeptide; and
(b) an isolated Smac polypeptide; wherein upon contact the introduced molecule binds to the c-IAP and enhances c-IAP auto-ubiquitylation activity, thereby causing the ubiquitylation-mediated, auto-degradation of the c-IAP.

20. The method of claim 19, wherein the nucleotide sequence is introduced into the cell by a vector, the vector comprising a Smac nucleotide sequence, an activator and a marker.

21. The method of claim 19, wherein the isolated Smac polypeptide is selected from the group consisting of a native polypeptide, a recombinant polypeptide, and a synthetic peptide.

22. The method of claim 19, wherein the isolated Smac consists of SEQ ID NO 1.

23. The method of claim 19, wherein the nucleotide sequence consists of SEQ ID NO 3.

24. The method of claim 19, wherein the c-IAP is c-IAP1 or c-IAP2.

25. The method of claim 19, wherein the c-IAP auto-ubiquitylation activity results from the E₃ activity of the c-IAP ring domain.

26. The method of claim 19, wherein the cell is a cultured cell.

27. The method of claim 19, wherein the cell is disposed within a living organism.

28. The method of claim 19, wherein the organism is a mammal.

29. The method of claim 28, wherein the mammal is a human.

30. The method of claim 19, wherein the Smac polypeptide binds to a BIR domain of the c-IAP through its N-terminal IAP-binding motif.

31. The method of claim 30, wherein the N-terminal IAP-binding motif is a polypeptide having the sequence of SEQ ID NO 2.

32. A method for the selective auto-degradation of a c-IAP in a cell having a plurality of IAP molecules, the method comprising introducing into the cell an effective amount of an isolated molecule selected from the group consisting of:
(a) a nucleotide sequence encoding a Smac polypeptide under conditions resulting in the expression of the Smac polypeptide; and
(b) an isolated Smac polypeptide; wherein upon contact the introduced molecule binds to the c-IAP and enhances c-IAP auto-ubiquitylation activity, thereby causing the ubiquitylation-mediated, auto-degradation of the c-IAP.

33. The method of claim 32, wherein the nucleotide sequence is introduced into the cell by a vector, the vector comprising a Smac nucleotide sequence, an activator and a marker.

34. The method of claim 32, wherein the isolated Smac polypeptide is selected from the group consisting of a recombinant peptide, a synthetic peptide, and a native polypeptide.

35. The method of claim 34, wherein the Smac polypeptide consists of SEQ ID NO 1.

36. The method of claim 32, wherein the nucleotide sequence consists of SEQ ID NO 3.

37. The method of claim 32, wherein the c-IAP is c-IAP1.

38. The method of claim 32, wherein the c-IAP is c-IAP2.

39. The method of claim 32, wherein the IAP molecule is a mammalian IAP.

40. The method of claim 32, wherein the IAP molecule is selected from the group consisting of c-IAP1, c-IAP2, XIAP and Livin.

41. The method of claim 32, wherein the c-IAP auto-ubiquitylation activity results from the E₃ activity of the c-IAP ring domain.

42. The method of claim 32, wherein the cell is a cultured cell.

43. The method of claim 32, wherein the cell is disposed within a living organism.

44. The method of claim 43, wherein the organism is a mammal.

45. The method of claim 44, wherein the mammal is a human.

46. The method of claim 32, wherein the Smac polypeptide binds to a BIR domain of the c-IAP through its N-terminal IAP-binding motif.

47. The method of claim 46, wherein the N-terminal IAP-binding motif is a polypeptide having the sequence of SEQ ID NO 2.

48. A method for promoting the E₃ activity of a c-IAP molecule, the method comprising contacting in vitro, the c-IAP molecule with a Smac polypeptide.

49. The method of claim 48, wherein the Smac polypeptide consists of SEQ ID NO 1.

50. The method of claim 48, wherein the Smac polypeptide is encoded by a nucleotide sequence consisting of SEQ ID NO 3.

51. The method of claim 48, wherein the Smac polypeptide binds to a BIR domain of the c-IAP through its N-terminal IAP-binding motif.

52. The method of claim 51, wherein the N-terminal IAP-binding motif is a polypeptide having the sequence of SEQ ID NO 2.

53. The method of claim 48, wherein the c-IAP is c-IAP1 or c-IAP2.

54. An isolated, ubiquilated molecule comprising a Smac polypeptide, a c-IAP, an ubiquitin, and a proteosome.

55. The molecule of claim 54, wherein the c-IAP is selected from the group consisting of c-IAP1 or c-IAP2.

56. An in vitro composition comprising an isolated Smac polypeptide, at least one c-IAP degradation product, and E₃.

57. The composition of claim 56, wherein the c-IAP is c-IAP1 or c-IAP2.

58. An isolated, ubiquilated molecule comprising a Smac 6 polypeptide having SEQ ID NO. 2, a c-IAP, an ubiquitin, and a proteosome.

59. The molecule of claim 58, wherein the c-IAP is c-IAP1 or c-IAP2.

60. An in vitro composition comprising an isolated Smac 6 polypeptide having SEQ ID NO. 2, at least one c-IAP degradation product, and E₃.

61. A kit comprising a Smac polypeptide having SEQ ID NO. 1, c-IAP1, c-IAP2, ubiquitin, E₁, E₂, and E₃.

62. A composition comprising a Hela cell transfected with a vector comprising a Smac nucleotide sequence having SEQ ID NO. 3 and a vector having a nucleotide sequence that encodes a c-IAP.

63. The composition of claim 62, wherein the c-IAP is selected from the group consisting of c-IAP1 and c-IAP2.

64. The composition of claim 63, wherein the vector having the Smac nucleotide sequence is pcDNA3.1 and the vector having the c-IAP nucleotide sequence is p3×FLAG-CMV-7.


SEQUENCE LISTING (3 sequences)

SEQ ID NO: 1 - protein, 239 aa, Homo sapiens
MAALKSWLSRSVTSFFRYRQCLCVPVVANFKKRCFSELIRPWHKTVTIGFGVTLCAVPIAQKSEPHSLSSEALMRRAVSLVTDSTSTFLSQTTYALIEAITEYTKAVYTLTSLYRQYTSLLGKMNSEEEDEVWQVIIGARAEMTSKHQEYLKLETTWMTAVGLSEMAAEAAYQTGADQASITARNHIQLVKLQVEEVHQLSRKAETKLAEAQIEELRQKTQEEGEERAESEQEAYLRED

SEQ ID NO: 2 - protein, 6 aa, Homo sapiens
AVPIAQ

SEQ ID NO: 3 - protein, 1356 aa, Homo sapiens
GGCGTCCGCGCGCTGCACAATGGCGGCTCTGAAGAGTTGGCTGTCGCGCAGCGTAACTTCATTCTTCAGGTACAGACAGTGTTTGTGTGTTCCTGTTGTGGCTAACTTTAAGAAGCGGTGTTTCTCAGAATTGATAAGACCATGGCACAAAACTGTGACGATTGGCTTTGGAGTAACCCTGTGTGCGGTTCCTATTGCACAGAAATCAGAGCCTCATTCCCTTAGTAGTGAAGCATTGATGAGGAGAGCAGTGTCTTTGGTAACAGATAGCACCTCTACCTTTCTCTCTCAGACCACATATGCGTTGATTGAAGCTATTACTGAATATACTAAGGCTGTTTATACCTTAACTTCTCTTTACCGACAATATACAAGTTTACTTGGGAAAATGAATTCAGAGGAGGAAGATGAAGTGTGGCAGGTGATCATAGGAGCCAGAGCTGAGATGACTTCAAAACACCAAGAGTACTTGAAGCTGGAAACCACTTGGATGACTGCAGTTGGTCTTTCAGAGATGGCAGCAGAAGCTGCATATCAAACTGGCGCAGATCAGGCCTCTATAACCGCCAGGAATCACATTCAGCTGGTGAAACTGCAGGTGGAAGAGGTGCACCAGCTCTCCCGGAAAGCAGAAACCAAGCTGGCAGAAGCACAGATAGAAGAGCTCCGTCAGAAAACACAGGAGGAAGGGGAGGAGCGGGCTGAGTCGGAGCAGGAGGCCTACCTGCGTGAGGATTGAGGGCCTGAGCACACTGCCCTGTCTCCCCACTCAGTGGGGAAAGCAGGGGCAGATGCCACCCTGCCCAGGGTTGGCATGACTGTCTGTGCACCGAGAAGAGGCGGCAGGTCCTGCCCTGGCCAATCAGGCGAGACGCCTTTGTGAGCTGTGAGTGCCTCCTGTGGTCTCAGGCTTGCGCTGGACCTGGTTCTTAGCCCTTGGGCACTGCACCCTGTTTAACATTTCACCCCACTCTGTACAGCTGCTCTTACCCATTTTTTTTACCTCACACCCAAAGCATTTTGCCTACCTGGGTCAGAGAGAGGAGTCCTTTTTGTCATGCCCTTAAGTTCAGCAACTGTTTAACCTGTTTTCAGTCTTATTTACGTCGTCAAAAATGATTTAGTACTTGTTCCCTCTGTTGGGATGCCAGTTGTGGCAGGGGGAGGGGAACCTGTCCAGTTTGTACGATTTCTTTGTATGTATTTCTGATGTGTTCTCTGATCTGCCCCCACTGTCCTGTGAGGACAGCTGAGGCCAAGGAGTGAAAAACCTATTACTACTAAGAGAAGGGGTGCAGAGTGTTTACCTGGTGCTCTCAACAGGACTTAACATCAACAGGACTTAACACAGAAAAA